(19) United States
(12) Patent Application Publication
(10) Pub. No.: US 2014/0187538 A1
(43) Pub. Date: Jul. 3, 2014

(54) LORCASERIN HYDROCHLORIDE

(71) Applicant: DR. REDDY'S LABORATORIES LTD., Hyderabad (IN)

(72) Inventors: Rajesham Boge, Hyderabad (IN); Arjun Kumar Tummala, Visakhapatnam (IN); Vishweshwar Peddy, Hyderabad (IN); Srinivasulu Rangineni, Mahabubnagar (IN); Vilas Hareshwar Dahanukar, Hyderabad (IN); Bandichhor Rakeshwar, Dist: Sultanpur (IN); Areveli Srinivas, Dist: Karimnagar (IN); Macharla Prabhakar, Mahabubnagar (IN); Varanasi Ganesh, Hyderabad (IN)

(73) Assignee: DR. REDDY'S LABORATORIES LTD., Hyderabad (IN)

(21) Appl. No.: 14/141,112

(22) Filed: Dec. 26, 2013

(30) Foreign Application Priority Data
Dec. 28, 2012 (IN) 5505/CHE/2012
Apr. 26, 2013 (IN) 1890/CHE/2013
Nov. 27, 2013 (IN) 5463/CHE/2013

(51) Int. Cl. A61K 9/10; A61K 31/55; C07D 223/16

(52) U.S. Cl. CPC A61K 9/10; C07D 223/16; A61K 31/55
USPC 514/217.01; 540/594

(57) ABSTRACT

The present invention relates to amorphous lorcaserin hydrochloride; amorphous solid dispersion comprising lorcaserin hydrochloride and one or more pharmaceutically acceptable carries; processes for preparation thereof; pharmaceutical compositions comprising amorphous lorcaserin hydrochloride and one or more pharmaceutically acceptable carries.

### FIELD OF INVENTION

The present application relates to processes for the preparation of lorcaserin and its intermediates. Present invention also relates to amorphous lorcaserin hydrochloride and amorphous solid dispersion comprising lorcaserin hydrochloride.

### BACKGROUND

Lorcaserin is chemically described as (R)-8-chloro-1-methyl-2,3,4,5-tetrahydro-1H-3-benzazepine. It has the chemical structure of formula (I).

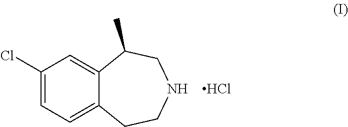

Lorcaserin hydrochloride is an agonist of the 5-HT_{2C} receptor and shows effectiveness at reducing obesity in animal models and humans. Further, 5-HT_{2C} receptor is recognized as a well-accepted receptor target for the treatment of obesity, psychiatric, and other disorders.

Various processes for the preparation of compound of Formula (I), its related salts, enantiomers and intermediates, have been reported in WO 2003/086306, WO 2005/019179, WO 2006/069363, WO 2007/120517, WO 2008/070111, WO 2009/111004 and WO2010/148207 each of which is incorporated herein by reference in its entirety.

In this application, there are provided simple, economical, cost effective, scalable and robust processes for the preparation of lorcaserin, intermediates thereof and salts thereof.

The occurrence of different solid forms is possible for some compounds. A single compound may give rise to a variety of solid forms having distinct physical properties. This variation in solid forms may be significant and may result in differences in pharmaceutical products with respect to solubility, bioavailability, stability and other properties. Because solid forms may vary in their physical properties, regulatory authorities require that efforts shall be made to identify all possible solid forms, e.g., crystalline, amorphous, solvated, etc., of new drug substances.

The existence and possible number of solid forms for a given compound cannot be predicted, and there are no “standard” procedures that may be used to prepare solid forms of a substance. However, new forms of a pharmaceutically useful compound may provide an opportunity to improve the performance characteristics of pharmaceutical products. For example, in some cases, different forms of the same drug may exhibit very different solubility and dissolution rates. The discovery of new solid forms enlarges selection of materials with which formulation scientists may design a pharmaceutically acceptable dosage form of a drug with a targeted release profile or other desired characteristics.

Amorphous form of lorcaserin hydrochloride resulted in drug substance with limited physical stability. For example, amorphous lorcaserin hydrochloride tends to crystallize over a period of time. It has been surprisingly found that preparing amorphous solid dispersion comprising lorcaserin hydrochloride possesses significant physical stability improvements over amorphous lorcaserin hydrochloride alone.

### SUMMARY

In first embodiment, the present invention provides a process for preparing a compound of formula (II) or salt thereof;

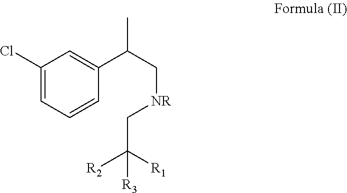

wherein R is H or an amino protecting group; and each of R₁, R₂ and R₃ is independently selected from H, halogen, alkyl, alkoxy, aryloxy, aralkyloxy, allyloxy, carboxy, alkoxy carbonyl, aryloxy carbonyl, aralkyloxy carbonyl.
which comprises one or more of the following steps:
(a) converting a compound of formula (III)

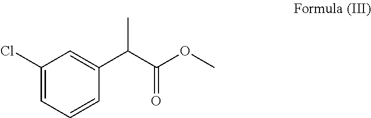

to provide a compound of formula (IV)

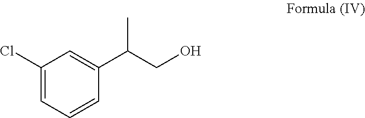

(b) converting compound of formula (IV) to compound of formula (V)

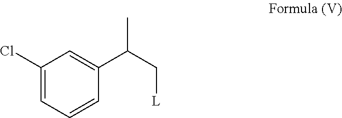

wherein ‘L’ is a leaving group,
(c) converting the compound of formula (V) to a compound of formula (II) or salt thereof.

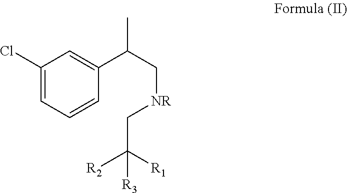

wherein R, R₁, R₂, and R₃ are as defined above.

In second embodiment, the present invention provides a process for preparing a compound of formula (II) or salt thereof;

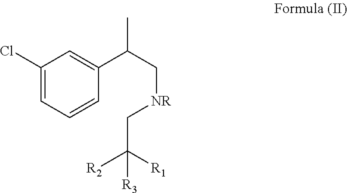

wherein R, R₁, R₂, and R₃ are as defined above, which comprises one or more of the following steps:
(a) converting a compound of formula (IV)

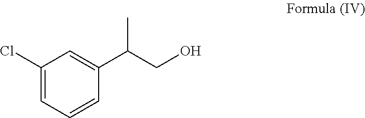

to a compound of formula (V)

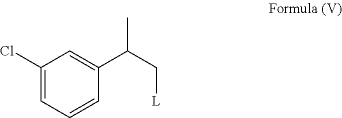

wherein L is a leaving group,
(b) converting the compound of formula (V) to a compound of formula (II) or salt thereof.

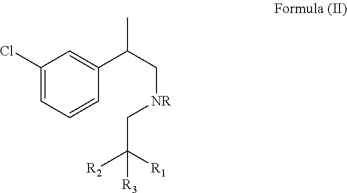

wherein R, R₁, R₂, and R₃ are as defined above.

In third embodiment, the present invention provides a process for preparing a compound of formula (II) or salt thereof;

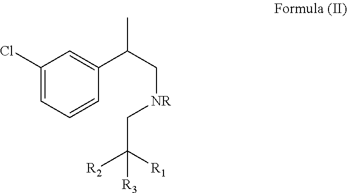

wherein R, R₁, R₂, and R₃ are as defined above, which comprises:
(a) converting the compound of formula (V) to a compound of formula (II) or salt thereof.

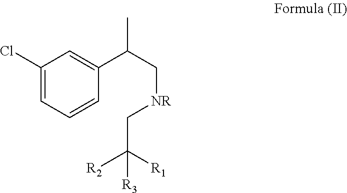

wherein R, R₁, R₂, and R₃ are as defined above.

In fourth embodiment, the present invention provides a process for preparing a compound of formula (VI) or salt thereof;

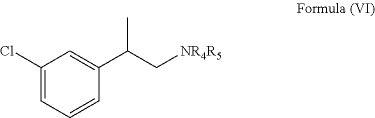

which comprises one or more of the following steps:
(a) converting the compound of formula (VII)

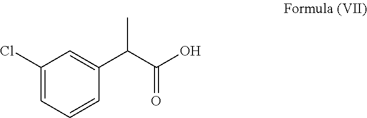

to a compound of formula (VIII)

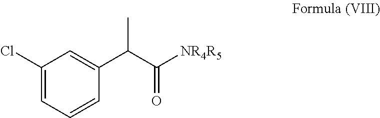

wherein R₄ and R₅ are as defined for R, R₁, R₂, and R₃ above,
(b) converting the compound of formula (VIII) to obtain a compound of formula (VI)

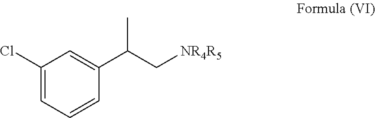

wherein R₄ and R₅ are as defined above.

In fifth embodiment, the present invention provides a process for preparing a compound of formula (VI), which comprises:

(a) converting the compound of formula (VIII) to a compound of formula (VI)

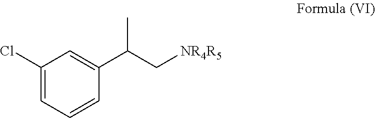

wherein R₄ and R₅ are as defined above.

In sixth embodiment, the present invention provides a process for preparing a compound of formula (VI), which comprises:

(a) converting the compound of formula (IX)

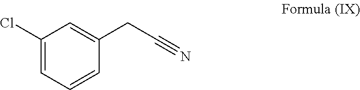

to a compound of formula (X)

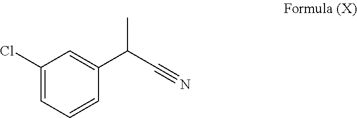

(b) converting the compound of formula (X) to form compound of formula (VI)

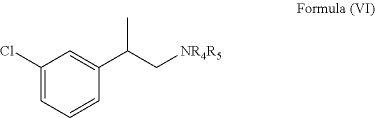

wherein R₄=R₅=H

In seventh embodiment, the present invention provides a process for preparing a compound of formula (VI), which comprises:

(a) converting the compound of formula (X)

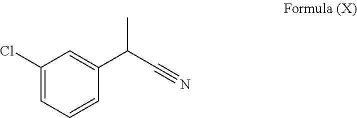

to compound of formula (VI)

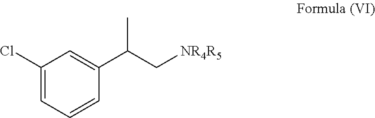

wherein R₄=R₅=H

In eighth embodiment, the present invention provides a process for preparing a compound of formula (VI), which comprises:

(a) converting the compound of formula (IV)

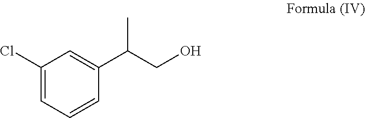

to compound of (XI)

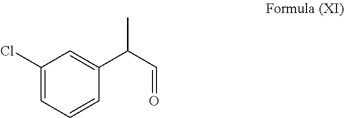

(b) converting the compound of formula (XI) to form compound of formula (VI)

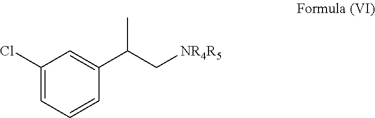

wherein R₄=R₅=H.

In ninth embodiment, the present invention provides a process for preparing a compound of formula (VI), which comprises:

(a) converting the compound of formula (XI)

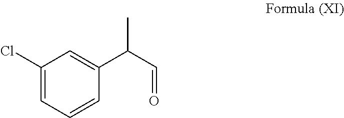

to a compound of formula (VI)

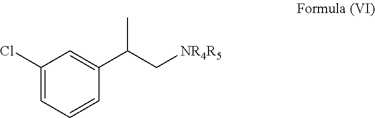

wherein R₄=R₅=H.

In tenth embodiment, the present invention provides a process for preparing a compound of formula (VI), which comprises:

(a) converting the compound of formula (III)

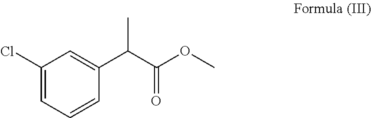

to a compound of formula (IV),

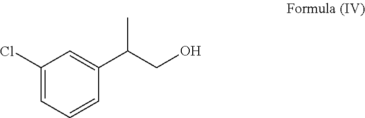

(b) converting the compound of formula (IV) to a compound of formula (V),

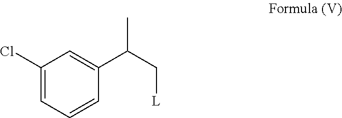

wherein L is a leaving group,
(c) converting the compound of formula (V) to a compound of formula (XII),

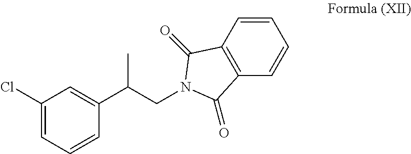 [SMILES: CC(CN1C(=O)c2ccccc2C1=O)c1cccc(Cl)c1]

(d) converting the compound Formula (XII) to compound of formula (VI)

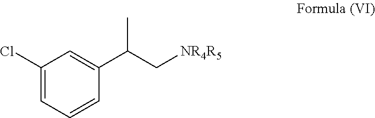

wherein R₄=R₅=H.

In eleventh embodiment, the present invention provides a process for preparing a compound of formula (VI), which comprises:

(a) converting the compound of formula (IV) to a compound of formula (V)

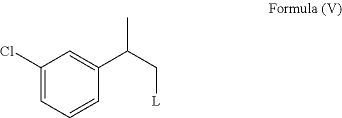

wherein ‘L’ is a leaving group,
(b) converting the compound of formula (V) to a compound of formula (XII)

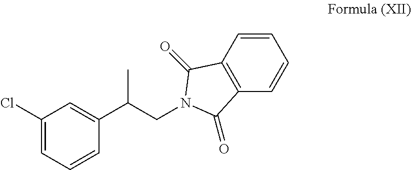 [SMILES: CC(CN1C(=O)c2ccccc2C1=O)c1cccc(Cl)c1]

(c) converting the compound Formula (XII) to compound of formula (VI)

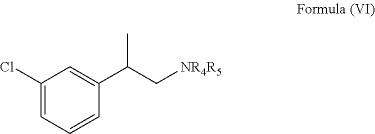

wherein R₄=R₅=H.

In twelfth embodiment, the present invention provides a process for preparing a compound of formula (VI), which comprises:

(a) converting the compound of formula (V) to a compound of formula (XII)

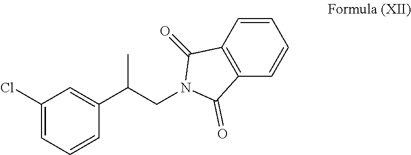 [SMILES: CC(CN1C(=O)c2ccccc2C1=O)c1cccc(Cl)c1]

(b) converting the compound Formula (XII) to a compound of formula (VI)

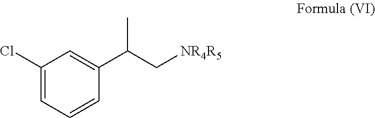

wherein R₄=R₅=H.

In thirteenth embodiment, the present invention provides a process for preparing a compound of formula (VI), which comprises:

(a) converting the compound Formula (XII)

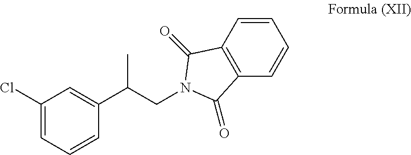 [SMILES: CC(CN1C(=O)c2ccccc2C1=O)c1cccc(Cl)c1]

to a compound of formula (VI)

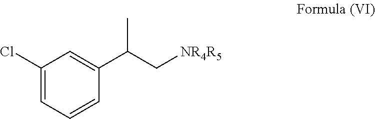

wherein R₄=R₅=H.

In fourteenth embodiment, the present invention provides a process for preparing a compound of formula (XIV), which comprises:

(a) converting the compound of formula (XIII)

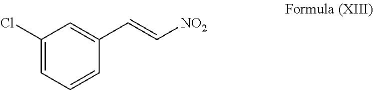 [SMILES: O=[N+]([O-])/C=C/c1cccc(Cl)c1]

to a compound of formula (XIV)

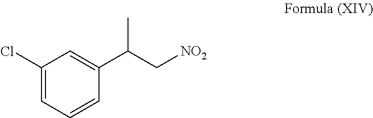

In fifteenth embodiment, the present invention provides a process for preparing a compound of formula (II) or salt thereof;

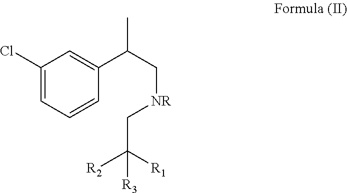

wherein R, R₁, R₂, and R₃ are as defined above.
which comprises one or more of the following steps:
(a) converting the compound of formula (XV)

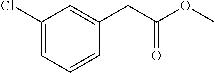

into a compound of formula (III)
(b) converting a compound of formula (III)

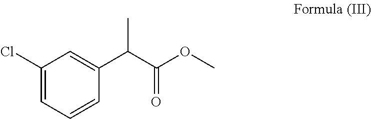

to provide a compound of formula (IV)

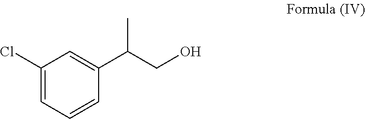

(c) converting compound of formula (IV) to compound of formula (V)

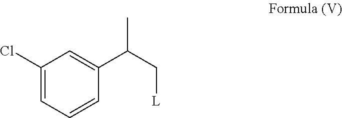

wherein L is a leaving group,
(d) converting the compound of formula (V) to a compound of formula (II) or salt thereof.

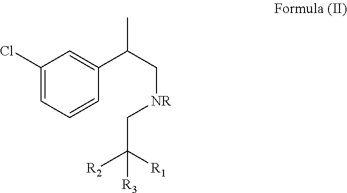

wherein R, R₁, R₂, and R₃ are as defined above.

In sixteenth embodiment, the present invention provides a process for preparing a compound of formula (VI) or salt thereof;

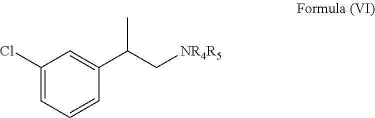

which comprises one or more of the following steps:
(a) converting the compound of (III)

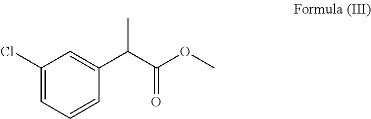

to compound of formula (VII)
(b) converting the compound of formula (VII)

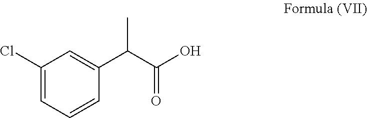

to a compound of formula (VIII)

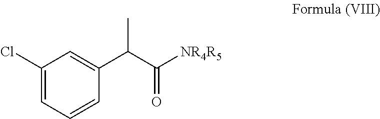

wherein R₄ and R₅ are as defined for R, R₁, R₂, and R₃ above,
(c) converting the compound of formula (VIII) to obtain a compound of formula (VI)

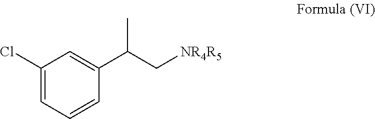

wherein R₄ and R₅ are as defined above.

In seventeenth, embodiment, the present invention provides a process for preparing a compound of formula (VI) or salt thereof;

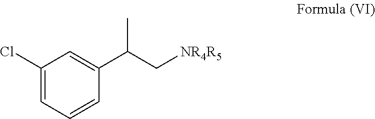

which comprises one or more of the following steps:
(a) converting the compound of formula (XV)

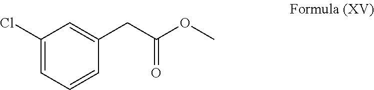

to a compound of formula (III)
(b) converting the compound of formula (III)

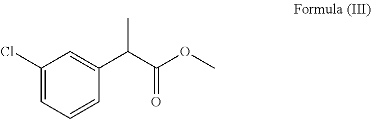

to compound of formula (VII)
(c) converting the compound of formula (VII)

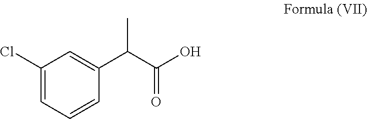

to a compound of formula (VIII)

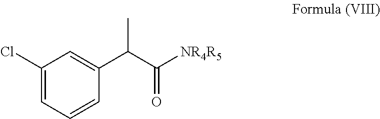

(d) converting the compound of formula (VIII) to obtain a compound of formula (VI)

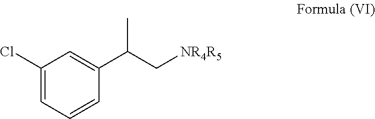

wherein R4 and R5 are as defined for R, R1, R2, and R3 above.

In eighteenth embodiment, the present invention provides a process for preparing a compound of formula (VI), which comprises:

(a) converting the compound of formula (III)

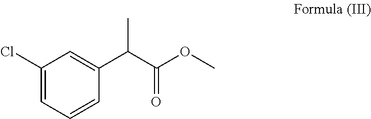

to the compound of formula (IV)
(b) converting the compound of formula (IV)

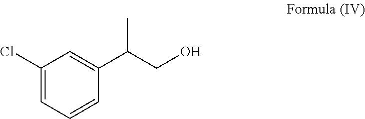

to a compound of (XI)

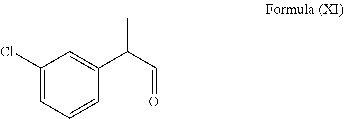

(c) converting the compound of formula (XI) to form compound of formula (VI)

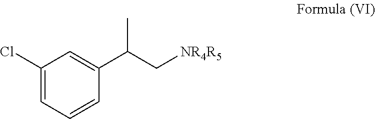

wherein R₄=R₅=H.

In nineteenth embodiment, the present invention provides a process for preparing a compound of formula (VI), which comprises:

(a) converting the compound of formula (XV)

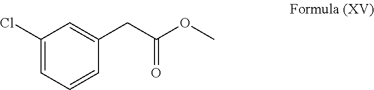

to compound of formula (III)

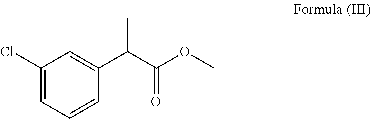

(b) converting the compound of formula (III)

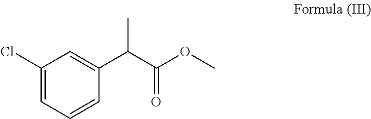

to the compound of formula (IV)
(c) converting the compound of formula (IV)

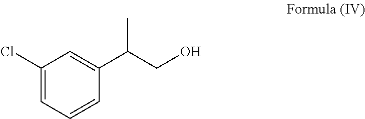

to compound of (XI)

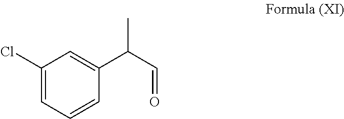

(d) converting the compound of formula (XI) to form compound of formula (VI)

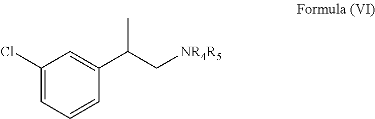

wherein R₄=R₅=H.

In twentieth embodiment, the present invention provides a process for preparing a compound of formula (VI), which comprises:

(a) converting the compound of formula (IV)

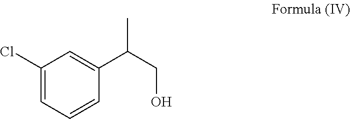

to a compound of formula (V)

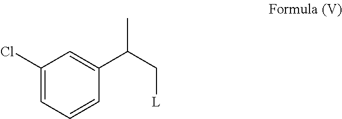

wherein L is a leaving group,
(b) converting the compound of formula (V) to a compound of formula (XII),

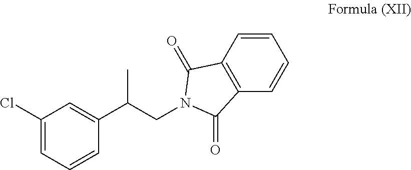 [SMILES: CC(CN1C(=O)c2ccccc2C1=O)c1cccc(Cl)c1]

(c) converting the compound Formula (XII) to compound of formula (VI)

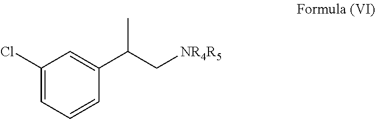

wherein R₄=R₅=H.

In twenty first embodiment, the present invention provides a process for preparing a compound of formula (VI), which comprises:

(a) converting the compound of formula (XV)

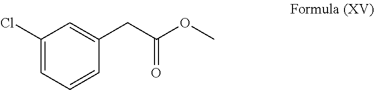

to a compound of formula (III)
(b) converting the compound of formula (III)

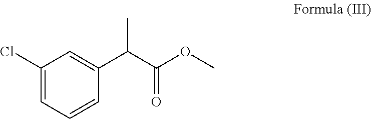

to a compound of formula (IV),

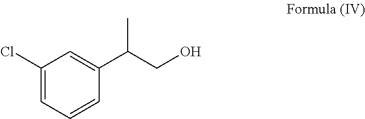

(c) converting the compound of formula (IV) to a compound of formula (V),

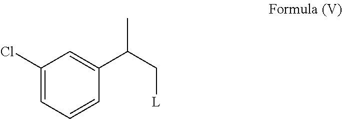

wherein L is a leaving group,
(d) converting the compound of formula (V) to a compound of formula (XII),

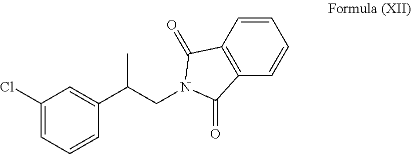 [SMILES: CC(CN1C(=O)c2ccccc2C1=O)c1cccc(Cl)c1]

(e) converting the compound Formula (XII) to compound of formula (VI)

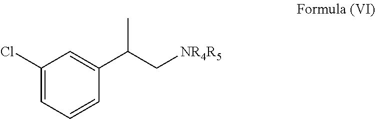

In twenty second embodiment, the present application provides an amorphous form of lorcaserin hydrochloride.

In twenty third embodiment, the present application provides an amorphous form of lorcaserin hydrochloride characterized by X-ray powder diffraction pattern substantially as illustrated by FIG. 2.

In twenty fourth embodiment, the present application provides a process for the preparation of amorphous form of lorcaserin hydrochloride, comprising:

a) providing a solution of lorcaserin hydrochloride in a solvent; and

b) isolating amorphous form of lorcaserin hydrochloride.

In twenty fifth embodiment, the present application provides pharmaceutical composition comprising amorphous form of lorcaserin hydrochloride and one or more pharmaceutically acceptable carriers.

In twenty sixth embodiment, the present application provides amorphous solid dispersion comprising lorcaserin hydrochloride and one or more pharmaceutically acceptable carriers.

In twenty seventh embodiment, the present application provides amorphous solid dispersion comprising lorcaserin hydrochloride and one or more pharmaceutically acceptable carriers characterized by X-ray powder diffraction pattern substantially as illustrated by FIG. 1, FIG. 3 or FIG. 4.

In twenty eighth embodiment, the present application provides a process for preparing an amorphous solid dispersion comprising lorcaserin hydrochloride and one or more pharmaceutically acceptable carriers, comprising the steps of:
a) dissolving or dispersing lorcaserin hydrochloride and one or more pharmaceutically acceptable carriers in a suitable solvent or mixture of solvents; and
b) isolating an amorphous solid dispersion comprising lorcaserin hydrochloride and one or more pharmaceutically acceptable carriers.

In twenty ninth embodiment, the present application provides a process for preparing an amorphous solid dispersion comprising lorcaserin hydrochloride and one or more pharmaceutically acceptable carriers, comprising the steps of:
(a) dissolving or dispersing lorcaserin hydrochloride under heating in one or more pharmaceutically acceptable carriers; and
(b) cooling down the mixture.

In thirtieth embodiment, the present application provides a process for preparing an amorphous solid dispersion comprising lorcaserin hydrochloride and one or more pharmaceutically acceptable carriers, comprising the steps of:
(a) dissolving or dispersing lorcaserin hydrochloride and one or more pharmaceutically acceptable carriers in a suitable solvent or mixture of solvents;
(b) spray drying the mixture to obtain the said amorphous solid dispersion.

In thirty first embodiment, the present application provides pharmaceutical composition comprising amorphous solid dispersion of lorcaserin hydrochloride and one or more pharmaceutically acceptable carriers.

### BRIEF DESCRIPTION OF THE DRAWINGS

FIG. 1 is an illustration of powder X-ray diffraction (PXRD) pattern of amorphous solid dispersion comprising lorcaserin hydrochloride prepared in accordance with Example

FIG. 2 is an illustration of powder X-ray diffraction (PXRD) pattern of amorphous lorcaserin hydrochloride prepared in accordance with Example 30.

FIG. 3 is an illustration of powder X-ray diffraction (PXRD) pattern of amorphous solid dispersion comprising lorcaserin hydrochloride prepared in accordance with Example 31.

FIG. 4 is an illustration of powder X-ray diffraction (PXRD) pattern of amorphous solid dispersion comprising lorcaserin hydrochloride prepared in accordance with Example

FIG. 5 is an illustration of powder X-ray diffraction (PXRD) pattern of amorphous solid dispersion comprising lorcaserin hydrochloride prepared in accordance with Example 33.

### DETAILED DESCRIPTION

The term “about” as used in the present invention preceding a number and referring to it, means any value which lies within the range of ±10%, preferably within a range of ±5%, more preferably within a range of ±2%, still more preferably within a range of ±1% of its value. For example “about 10” should be construed as meaning within the range of 9 to 11, preferably within the range of 9.5 to 10.5, more preferably within the range of 9.8 to 10.2, and still more preferably within the range of 9.9 to 10.1.

Room temperature as used herein refers to ‘the temperatures of the thing close to or same as that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight”. This refers to a time interval, e.g., for the process or step, that spans the time during the night, when that process or step may not be actively observed. This time interval is from about 8 to about 18 hours, typically about 16 hours.

Unless indicated, the solid state forms of the present invention may be dried. Drying may be carried out, for example, at elevated temperature with or without reduced pressure.

Drying may be suitably carried out in a tray dryer, vacuum oven, Büchi® Rotavapor®, air oven, fluidized bed dryer, spin flash dryer, flash dryer, cone dryer, agitated nutsche filter cum dryer, nauta dryer or the like or any other suitable dryer.

The drying may be carried out at temperature of less than about 150° C., or less than about 120° C., or less than about 100° C., or less than about 70° C., or less than about 60° C., or less than about 50° C., or less than about 40° C., or less than about 20° C., or less than about 0° C., or less than about −20° C. or any other suitable temperature. The drying may be carried out under reduced pressure, that is, less than standard atmospheric pressure or at atmospheric pressure or any other suitable pressure. The drying may take place over a period of about 30 minutes to about 12 hours, or about 2 hours to about 4 hours, or any other suitable time period.

The dried product may be optionally subjected to techniques such as sieving to get rid of lumps before or after drying. The dried product may be optionally milled to get desired particle sizes. Milling or micronization may be performed before drying, or after the completion of drying of the product. Techniques that may be used for particle size reduction include, without limitation, ball, roller and hammer mills, and jet mills.

The reactions of the processes described herein can be carried out in air or under an inert atmosphere. Typically, reactions containing reagents or products that are substantially reactive with air can be carried out using air-sensitive synthetic techniques that are well known to the person skilled in art.

A solid that is in the “amorphous” solid state form means that it is in a non-crystalline state. Amorphous solids generally possess crystal-like short range molecular arrangement, but no long range order of molecular packing as are found in crystalline solids. The solid state form of amorphous solid can be determined by X-Ray Powder Diffraction (XPRD), or other standard techniques known to those skilled in the art.

In general, the term “solid dispersion” refers to a system in a solid state comprising at least two components, wherein one component is dispersed throughout the other component or components.

The term “amorphous solid dispersion” as used herein, refers to solid dispersion which is substantially amorphous, that is, at least 80%, preferably at least 90%, most preferably at least 95%, is in amorphous form as determined by X-Ray Powder

Diffraction (XPRD). Lorcaserin hydrochloride used for the preparation of amorphous solid dispersion can be crystalline or amorphous.

An “alcohol solvent” is an organic solvent containing a carbon bound to a hydroxyl group. “Alcoholic solvents” include, but are not limited to, methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, C₁₋₆ alcohols, or mixtures thereof.

An “aliphatic or alicyclic hydrocarbon solvent” refers to a liquid, non-aromatic, hydrocarbon, which may be linear, branched, or cyclic. It is capable of dissolving a solute to form a uniformly dispersed solution. Examples of a hydrocarbon solvents include, but are not limited to, n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, C₅-C₈ aliphatic hydrocarbons, petroleum ethers, or mixtures thereof.

“Aromatic hydrocarbon solvent” refers to a liquid, unsaturated, cyclic, hydrocarbon containing one or more rings which has at least one 6-carbon ring containing three double bonds. It is capable of dissolving a solute to form a uniformly dispersed solution. Examples of aromatic hydrocarbon solvents include, but are not limited to, benzene toluene, ethylbenzene, m-xylene, o-xylene, p-xylene, indane, naphthalene, tetralin, trimethylbenzene, chlorobenzene, fluorobenzene, trifluorotoluene, anisole, C6-C10 aromatic hydrocarbons, or mixtures thereof.

An “ester solvent” is an organic solvent containing a carboxyl group —(C═O)—O— bonded to two other carbon atoms. “Ester solvents” include, but are not limited to, ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate, C₃₋₆ esters, or mixtures thereof.

A “halogenated hydrocarbon solvent” is an organic solvent containing a carbon bound to a halogen. “Halogenated hydrocarbon solvents” include, but are not limited to, dichloromethane, 1,2-dichloroethane, trichloroethylene, perchloroethylene, 1,1,1-trichloroethane, 1,1,2-trichloroethane, chloroform, carbon tetrachloride, or mixtures thereof.

A “ketone solvent” is an organic solvent containing a carbonyl group —(C═O)— bonded to two other carbon atoms. “Ketone solvents” include, but are not limited to, acetone, ethyl methyl ketone, diethyl ketone, methyl isobutyl ketone, C₃₋₆ ketones, 4-methyl-pentane-2-one or mixtures thereof.

A “nitrile solvent” is an organic solvent containing a cyano —(C≡N) bonded to another carbon atom. “Nitrile solvents” include, but are not limited to, acetonitrile, propionitrile, C₂₋₆ nitriles, or mixtures thereof.

A “polar aprotic solvent” has a dielectric constant greater than 15 and is at least one selected from the group consisting of amide-based organic solvents, such as N,N-dimethylformamide (DMF), N,N-dimethylacetamide (DMA), N-methylpyrrolidone (NMP), formamide, acetamide, propanamide, hexamethyl phosphoramide (HMPA), and hexamethyl phosphorus triamide (HMPT); nitro-based organic solvents, such as nitromethane, nitroethane, nitropropane, and nitrobenzene; pyridine-based organic solvents, such as pyridine and picoline; sulfone-based solvents, such as dimethylsulfone, diethylsulfone, diisopropylsulfone, 2-methylsulfolane, 3-methylsulfolane, 2,4-dimethylsulfolane, 3,4-dimethy sulfolane, 3-sulfolene, and sulfolane; and sulfoxide-based solvents such as dimethylsulfoxide (DMSO).

An “ether solvent” is an organic solvent containing an oxygen atom —O— bonded to two other carbon atoms. “Ether solvents” include, but are not limited to, diethyl ether, diisopropyl ether, methyl t-butyl ether, glyme, diglyme, tetrahydrofuran, 2-methyltetrahydrofuran, 1,4-dioxane, dibutyl ether, dimethylfuran, 2-methoxyethanol, 2-ethoxyethanol, anisole, C₂₋₆ ethers, or the like.

As used herein, “comprising” (open-ended) means the element or elements recited, or their equivalents in structure or function, plus any other element or elements which are not recited. The terms “having” and “including” are also to be construed as open-ended. As used herein, “consisting essentially of” means that the application may include elements in addition to those recited in the claim, but only if the additional elements do not materially alter the basic and novel characteristics. All ranges recited herein include the endpoints, including those that recite a range “between” two values. Whether so indicated or not, all values recited herein are approximate as defined by the circumstances, including the degree of expected experimental error, technique error, and instrument error for a given technique used to measure a value.

Step (a) of the first embodiment, step (a) of the tenth embodiment and step (a) of the eighteenth embodiment may be affected in presence of suitable reducing agent. Suitable reducing reagent that may be used includes but not limited to alkali metal hydrides, such as lithium aluminum hydride, sodium borohydride, sodium dihydro-bis-(2-methoxyethoxy) aluminate solution (VITRIDE®), diisobutyl aluminium hydride, sodium cyanoborohydride or the like; sodium dithionite in alkaline medium; any combination thereof; or any other suitable reducing agent known in the art.

Step (a) of the first embodiment, step (a) of the tenth embodiment and step (a) of the eighteenth embodiment may be carried out in a suitable solvent. Suitable solvents that may be used include, but are not limited to: water, alcohols, such as for example, methanol, ethanol, propanol, butanol, pentanol, ethylene glycol, glycerol, or the like; ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; or any mixtures thereof.

Suitable temperatures for the reaction of step (a) of the first embodiment, step (a) of the tenth embodiment and step (a) of the eighteenth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

Step (a) of the first embodiment, step (a) of the tenth embodiment and step (a) of the eighteenth embodiment may be optionally carried out in an inert atmosphere such as nitrogen or argon.

The reaction mixture obtained in step (a) of the first embodiment, step (a) of the tenth embodiment and step (a) of the eighteenth embodiment may be optionally filtered to remove any insoluble solids, or particles may be removed by methods such as decantation, centrifugation, gravity filtration, suction filtration, or any other technique for the removal of solids.

The product of step (a) of the first embodiment, step (a) of the tenth embodiment and step (a) of the eighteenth embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation of compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound is not degraded in quality, at atmospheric pressure or under a reduced pressure. The drying may be carried out for any desired times until the required purity is achieved. For example, it may vary from about 1 hour to about 10 hours, or longer.

Optionally the product of step (a) of the first embodiment, step (a) of the tenth embodiment and step (a) of the eighteenth embodiment may be used directly in the next step without isolation.

Step (b) of the first embodiment, step (a) of the second embodiment, step (b) of the tenth embodiment and step (a) of the twentieth embodiment may be affected in presence of leaving group. Suitable leaving groups that may be used include but not limited to mesyl, tosyl, chlorine, bromine, iodine, alkylsulphonyloxy groups, arylsulphonyloxy groups, —O-methyl, —SCN, unsubstituted and fluorinated C₁-C₄-alkylsulfonyloxy, trialkylsilyl.

Step (b) of the first embodiment, step (b) of the tenth embodiment, step (a) of the second embodiment, step (a) of the ninth embodiment, step (a) of the fifteenth embodiment, step (a) of sixteenth embodiment, step (a) of the seventeenth embodiment, step (a) of the nineteenth embodiment, step (a) of the twentieth embodiment, step (c) of the first embodiment and step (a) of the twenty first embodiment may be optionally affected in presence of a base. Suitable bases that may be used include, but are not limited to: organic bases, such as for example, triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, or the like; inorganic bases, such as for example, alkali metal hydrides, such as for example, lithium hydride, sodium hydride, potassium hydride, or the like; sodamide; n-butyl lithium; lithium diisopropylamide; alkali metal hydroxides, such as for example, lithium hydroxide, sodium hydroxide, potassium hydroxide, and cesium hydroxide; alkaline metal hydroxides, such as for example, aluminum hydroxide, magnesium hydroxide, calcium hydroxide, or the like; alkoxides such as sodium methoxide, potassium tertiary butoxide or the like; alkali metal carbonates, such as for example, sodium carbonate, potassium carbonate, lithium carbonate, cesium carbonate, or the like; alkaline earth metal carbonates, such as for example, magnesium carbonate, calcium carbonate, or the like; alkali metal bicarbonates, such as for example, lithium bicarbonate, sodium bicarbonate, potassium bicarbonate, or the like; and ion exchange resins including resins bound to ions, such as for example, sodium, potassium, lithium, calcium, magnesium, substituted or unsubstituted ammonium ions, or the like; or any other suitable bases.

Step (b) of the first embodiment, step (b) of the tenth embodiment, step (a) of the second embodiment, step (a) of the ninth embodiment, step (a) of the twentieth embodiment, step (a) of the fifteenth embodiment, step (a) of sixteenth embodiment, step (a) of the seventeenth embodiment, step (a) of the nineteenth embodiment and step (a) of the twenty first embodiment may be carried out in a suitable solvent. Suitable solvents that may be used include, but are not limited to: ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, methyl tetrahydrofuran, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N,N-dimethylacetamide, N-methylpyrrolidone, pyridine, dimethylsulphoxide, sulpholane, formamide, acetamide, propanamide, or the like; nitromethane; or any mixtures thereof.

Suitable temperature for the reaction of Step (b) of the first embodiment, step (b) of the tenth embodiment, step (a) of the second embodiment, step (a) of the ninth embodiment, step (a) of the twentieth embodiment, step (a) of the fifteenth embodiment, step (a) of sixteenth embodiment, step (a) of the seventeenth embodiment, step (a) of the nineteenth embodiment and step (a) of the twenty first embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

The reaction mixture obtained in Step (b) of the first embodiment, step (b) of the tenth embodiment, step (a) of the second embodiment, step (a) of the ninth embodiment, step (a) of the twentieth embodiment, step (a) of the fifteenth embodiment, step (a) of sixteenth embodiment, step (a) of the seventeenth embodiment, step (a) of the nineteenth embodiment and step (a) of the twenty first embodiment may be optionally filtered to remove any insoluble solids, or particles may be removed by methods such as decantation, centrifugation, gravity filtration, suction filtration, or any other suitable technique for the removal of solids.

The product of step (b) of the first embodiment, step (b) of the tenth embodiment, step (a) of the second embodiment, step (a) of the ninth embodiment, step (a) of the twentieth embodiment, step (a) of the fifteenth embodiment, step (a) of sixteenth embodiment, step (a) of the seventeenth embodiment, step (a) of the nineteenth embodiment and step (a) of the twenty first embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation compound of include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound, at atmospheric pressure or under a reduced pressure. The drying may be carried out for any desired times until the required purity is achieved. For example, it may vary from about 1 hour to about 10 hours, or longer.

Optionally the product in step (b) of the first embodiment, step (b) of the tenth embodiment, step (a) of the second embodiment, step (a) of the ninth embodiment, step (a) of the twentieth embodiment, step (a) of the fifteenth embodiment, step (a) of sixteenth embodiment, step (a) of the seventeenth embodiment, step (a) of the nineteenth embodiment and step (a) of the twenty first embodiment obtained may be used directly in the next step without isolation.

The conversion in step (c) of the first embodiment, step (b) of the second embodiment and step (a) of the third embodiment may be affected for example by reacting a compound of formula (V) with a compound of formula (XVI)

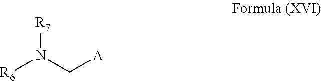

### Wherein:

R₆ is selected from H, X, CH₃COO⁻, tert-butoxycarbonyl, benzyloxycarbonyl, P-methoxybenzyloxy carbonyl, isopropyl or the like; R₇ is selected from H, isopropyl; A is selected from —COOH, —CHO, —COX, —C(OMe)₃, —C(OEt)₃, —CH(OMe)₂, —CH(OEt)₂, —CH₂OMe, —CH₂OEt, —CH₂X, —CH₂OH, —CH₃—CH₂L; wherein L is a leaving group as defined above; and X is selected from chlorine, bromine or iodine.

Step (c) of the first embodiment, step (b) of the second embodiment and step (a) of the third embodiment may be carried out in a suitable solvent. Suitable solvent that may be used includes, but not limited to: alcohols, such as for example, methanol, ethanol, propanol, butanol, pentanol, ethylene glycol, glycerol, or the like; ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, methyl tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N,N-dimethylacetamide, N-methylpyrrolidone, pyridine, dimethylsulphoxide, sulpholane, formamide, acetamide, propanamide, or the like; nitromethane; triethylamine; or any mixtures thereof.

Suitable temperatures for the reaction of step (c) of the first embodiment, step (b) of the second embodiment and step (a) of the third embodiment may be less than about 180° C., less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

The reaction mixture obtained in step (c) of the first embodiment, step (b) of the second embodiment and step (a) of third embodiment may be optionally filtered to remove any insoluble solids, or particles may be removed by methods such as decantation, centrifugation, gravity filtration, suction filtration, or any other technique for the removal of solids.

The product of step (c) of the first embodiment, step (b) of the second embodiment and step (a) of the third embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound, at atmospheric pressure or under a reduced pressure. The drying may be carried out for any desired times until the required purity is achieved. For example, it may vary from about 1 hour to about 10 hours, or longer.

Optionally the product obtained in step (c) of the first embodiment, step (b) of the second embodiment and step (a) of the third embodiment may be used directly in the next step without isolation.

Step (a) of the fourth embodiment may be affected in presence of in presence of compound of formula (XVI)

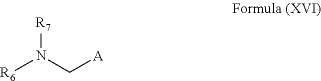

### Wherein:

R₆ is selected from H, X, CH₃COO⁻, tert-butoxycarbonyl, benzyloxycarbonyl, P-methoxybenzyloxy carbonyl, isopropyl or the like; R₇ is selected from H, isopropyl; A is selected from —COOH, —CHO, —COX, —C(OMe)₃, —C(OEt)₃, —CH(OMe)₂, —CH(OEt)₂, —CH₂OMe, —CH₂OEt, —CH₂X, —CH₂OH, —CH₃—CH₂L; wherein L is a leaving group as defined above; and X is selected from chlorine, bromine or iodine.

Step (a) of the fourth embodiment may be affected in presence of leaving group. Suitable leaving groups that may be used includes but not limited to mesyl, tosyl, chlorine, bromine, iodine, alkylsulphonyloxy groups, arylsulphonyloxy groups, —O-methyl, —SCN, unsubstituted and fluorinated C1-C4-alkylsulfonyloxy, trialkylsilyl.

Step (a) of the fourth embodiment may be affected in presence of base. Suitable bases that may be used include, but are not limited to: organic bases, such as for example, triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, ammonia or the like; inorganic bases, such as for example, alkali metal hydrides, such as for example, lithium hydride, sodium hydride, potassium hydride, or the like; sodamide; n-butyl lithium; lithium diisopropylamide; alkoxides such as sodium methoxide, potassium tertiary butoxide or the like; alkali metal carbonates, such as for example, sodium carbonate, potassium carbonate, lithium carbonate, cesium carbonate, or the like; alkaline earth metal carbonates, such as for example, magnesium carbonate, calcium carbonate, or the like; alkali metal bicarbonates, such as for example, lithium bicarbonate, sodium bicarbonate, potassium bicarbonate, or the like; and ion exchange resins including resins bound to ions, such as for example, sodium, potassium, lithium, calcium, magnesium, substituted or unsubstituted ammonium ions, or the like; or any other suitable bases.

Step (a) of the fourth embodiment may be carried out in a suitable solvent. Suitable solvents that may be used include, but are not limited to: alcohols such as for example, methanol, ethanol, propanol, butanol, pentanol, ethylene glycol, glycerol, or the like; ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N,N-dimethylacetamide, N-methylpyrrolidone, pyridine, dimethylsulphoxide, sulpholane, formamide, acetamide, propanamide, or the like; nitromethane; or any mixtures thereof.

Suitable temperatures used for the reaction of step (a) of the fourth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C. or any other suitable temperatures.

The reaction mixture obtained in step (a) of the fourth embodiment may be optionally filtered to remove any insoluble solids, or particles may be removed by methods such as decantation, centrifugation, gravity filtration, suction filtration, or any other technique for the removal of solids.

The product of step (a) of the fourth embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound is not degraded in quality, at atmospheric pressure or under a reduced pressure. The drying may be carried out for any desired times until the required purity is achieved. For example, it may vary from about 1 hour to about 10 hours, or longer.

Optionally the product of step (a) of the fourth embodiment may be used directly in the next step without isolation.

Step (b) of the fourth embodiment and step (a) of the fifth embodiment may be affected in presence of suitable reducing agents. Suitable reducing agents include but are not limited to catalytic hydrogenation using palladium-on-carbon, platinum(IV) oxide, or Raney™ nickel, or the like; metal mediated reduction such as zinc and acetic acid, zinc and hydrochloric acid, tin and hydrochloric acid, sodium amalgam in ethanol, or iron and acetic acid; tin chloride (II), titanium (III) chloride, or the like; alkali metal hydrides, such as lithium aluminum hydride, sodium borohydride, sodium dihydro-bis-(2-methoxyethoxy) aluminate solution (VITRIDE®), diisobutyl aluminium hydride, sodium cyanoborohydride or the like; sodium dithionite in alkaline medium; borane-tetrahydrofuran, borane dimethylsulfide, any combination thereof; or any other suitable reducing agent known in the art. Optionally reaction may be carried out under an atmosphere of hydrogen.

Step (b) of the fourth embodiment and step (a) of the fifth embodiment may be affected in presence of suitable lewis acids. Suitable lewis acids include boron containing reagents, aluminium containing reagent but, are not limited to BF₃: TBME (t-butyl methyl ether); BF₃:OEt₂; BF₃:O(CH₂CH₂CH₂CH₃)₂; BF₃:THF; and the like; alkyl aluminum halides, dialkyl aluminum halides, trialkyl aluminum, and aluminum halides (e.g., AlCl₃ and AlBr₃); transition metal ions or any other suitable reagent known in the art.

Step (b) of the fourth embodiment and step (a) of the fifth embodiment may be carried out in a suitable solvent. Suitable solvents that may be used include, but are not limited to: alcohols, such as for example, methanol, ethanol, propanol, butanol, pentanol, ethylene glycol, glycerol, or the like; ethers, such as for example, diethyl ether, diisopropyl ether, t-butyl methyl ether, dibutyl ether, tetrahydrofuran, methyl tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; or any mixtures thereof.

Suitable temperatures for the reaction of step (b) of the fourth embodiment and step (a) of the fifth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −50° C. or any other suitable temperatures.

The reaction mixture obtained in step (b) of the fourth embodiment and step (a) of the fifth embodiment may be optionally filtered to remove any insoluble solids, or particles may be removed by methods such as decantation, centrifugation, gravity filtration, suction filtration, or any other technique for the removal of solids.

The product of step (b) of the fourth embodiment and step (a) of the fifth embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound is not degraded in quality, at atmospheric pressure or under a reduced pressure. The drying may be carried out for any desired times until the required purity is achieved. For example, it may vary from about 1 hour to about 10 hours, or longer.

Optionally the product obtained in the step (b) of the fourth embodiment and step (a) of the fifth embodiment may be used directly in the next step without isolation.

Step (a) of the sixth embodiment may be affected in presence of suitable methylating agent but not limited to methyl iodide, dimethyl sulfate, dimethyl carbonate or the like.

Step (a) of the sixth embodiment may be affected in presence of suitable base. Suitable bases include, but not limited to organic bases, such as for example, triethylamine, tributylamine, N-methylmorpholine, N,N-diisopropylethylamine, N-methylpyrrolidine, pyridine, 4-(N,N-dimethylamino)pyridine, morpholine, imidazole, 2-methylimidazole, 4-methylimidazole, or the like; inorganic bases, such as for example, alkali metal hydrides, such as for example, lithium hydride, sodium hydride, potassium hydride, or the like; sodamide; n-butyl lithium; lithium diisopropylamide; lithium bis(trimethylsilyl)amide alkali metal hydroxides, such as for example, lithium hydroxide, sodium hydroxide, potassium hydroxide, and cesium hydroxide; alkali metal carbonates, such as for example, sodium carbonate, potassium carbonate, lithium carbonate, cesium carbonate, or the like; alkaline earth metal carbonates, such as for example, magnesium carbonate, calcium carbonate, or the like; alkali metal bicarbonates, such as for example, lithium bicarbonate, sodium bicarbonate, potassium bicarbonate, or the like; and ion exchange resins including resins bound to ions, such as for example, sodium, potassium, lithium, calcium, magnesium, substituted or unsubstituted ammonium ions, or the like; or any other suitable bases.

Step (a) of the sixth embodiment may be carried out in a suitable solvent. Suitable solvents that may be used include, but are not limited to: alcohols, such as for example, methanol, ethanol, propanol, butanol, pentanol, ethylene glycol, glycerol, or the like; ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N,N-dimethylacetamide, N-methylpyrrolidone, pyridine, dimethylsulphoxide, sulpholane, formamide, acetamide, propanamide, or the like; nitromethane; or any mixtures thereof.

Suitable temperatures for the reaction of (a) of the sixth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −50° C. or any other suitable temperatures.

The reaction mixture obtained in step (a) of the sixth embodiment may be optionally filtered to remove any insoluble solids, or particles may be removed by methods such as decantation, centrifugation, gravity filtration, suction filtration, or any other technique for the removal of solids.

The product of step (a) of the sixth embodiment may be isolated directly from the reaction mixture itself after the reaction is complete in step (a), or after conventional work up with techniques such as filtration, quenching with a suitable reagent, extraction or the like.

The isolation in step (a) of the sixth embodiment may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Optionally the product obtained in the step (a) of the sixth embodiment may be used directly in the next step without isolation.

Step (b) of the sixth embodiment and step (a) of the seventh embodiment may be affected in presence of reducing agent. Suitable reducing agents include, but not limited to catalytic hydrogenation using palladium-on-carbon, platinum(IV) oxide, or Raney™ nickel, or the like; metal mediated reduction such as zinc and acetic acid, zinc and hydrochloric acid, tin and hydrochloric acid, sodium amalgam in ethanol, or iron and acetic acid; tin chloride (II), titanium (III) chloride, or the like; alkali metal hydrides, such as lithium aluminum hydride, sodium borohydride, sodium dihydro-bis-(2-methoxyethoxy) aluminate solution (VITRIDE®), diisobutyl aluminium hydride, sodium cyanoborohydride or the like; sodium dithionite in alkaline medium; metal borides such as cobalt (II) boride, nickel(II) boride or the like. Lithium aluminium hydride-AlCl₃ or any other combination thereof; or any other suitable reducing agent known in the art. Optionally reaction may be carried out under an atmosphere of hydrogen.

Step (b) of the sixth embodiment and step (a) of the seventh embodiment may be carried out in a suitable solvent. Suitable solvents that may be used include, but are not limited to: alcohols, such as for example, methanol, ethanol, propanol, butanol, pentanol, ethylene glycol, glycerol, or the like; ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N, N-dimethylacetamide, N-methylpyrrolidone, pyridine, dimethylsulphoxide, sulpholane, formamide, acetamide, propanamide, or the like; nitromethane; acetic acid; or any mixtures thereof.

Step (b) of the sixth embodiment and step (a) of the seventh embodiment may be carried out in presence of reagent like acetic anhydride, di-tert-butyl dicarbonate or the like.

Suitable temperatures for the reaction in step (b) of the sixth embodiment and step (a) of the seventh embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −50° C. or any other suitable temperatures.

The reaction mixture obtained in step (b) of the sixth embodiment and step (a) of the seventh embodiment may be optionally filtered to remove any insoluble solids, or particles may be removed by methods such as decantation, centrifugation, gravity filtration, suction filtration, or any other technique for the removal of solids.

The product of step (b) of the sixth embodiment and step (a) of the seventh embodiment may be isolated directly from the reaction mixture itself after the reaction is complete, or after conventional work up with techniques such as filtration, quenching with a suitable reagent, extraction or the like.

The isolation in step (b) of the sixth embodiment and step (a) of the seventh embodiment may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Optionally the product obtained in the step (b) of the sixth embodiment and step (a) of the seventh embodiment may be used directly in the next step without isolation.

Step (a) of the eighth embodiment may be affected in presence of oxidizing agent. Suitable oxidizing agent includes but not limited to iodinated compounds such as 1,1,1-Triacetoxy-1,1-dihydro-1,2-benziodoxol-3(1H)-one (Dess-Martin periodinane), 2-iodoxybenzoic acid, chromium-based reagents such as collins reagent, PDC, PCC, activated DMSO, carbodiimide or the like.

Step (a) of the eighth embodiment may be carried out in a suitable solvent. Suitable solvents that may be used include, but are not limited to: ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N,N-dimethylacetamide, N-methylpyrrolidone, pyridine, dimethylsulphoxide, sulpholane, formamide, acetamide, propanamide, or the like; nitromethane; or any mixtures thereof.

Suitable temperatures for the reaction of step (a) of the eighth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

The reaction mixture obtained in step (a) of the eighth embodiment may be optionally filtered to remove any insoluble solids, or particles may be removed by methods such as decantation, centrifugation, gravity filtration, suction filtration, or any other technique for the removal of solids.

The product of step (a) of the eighth embodiment may be isolated directly from the reaction mixture itself after the reaction is complete in step (a), or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound, at atmospheric pressure or under a reduced pressure. The drying may be carried out for any desired times until the required purity is achieved. For example, it may vary from about 1 hour to about 10 hours, or longer.

Optionally the product obtained in the step (a) of the eighth embodiment may be used directly in the next step without isolation.

Step (b) of the eighth embodiment and step (a) of the ninth embodiment may be affected in presence of ammonium salts such as ammonium formate, ammonium acetate or the like; hydrazine, hydroxyl amine; ammonia gas or any other suitable reagent known in the art.

Step (b) of the eighth embodiment and step (a) of the ninth embodiment may be optionally accompanied with reduction reaction by catalytic hydrogenation using palladium-on-carbon, platinum(IV) oxide, or Raney™ nickel, or the like; metal mediated reduction such as zinc and acetic acid, zinc and hydrochloric acid, tin and hydrochloric acid, sodium amalgam in ethanol, or iron and acetic acid; tin chloride (II), titanium (III) chloride, or the like; alkali metal hydrides, such as lithium aluminum hydride, sodium borohydride, sodium dihydro-bis-(2-methoxyethoxy) aluminate solution (VITRIDE®), diisobutyl aluminium hydride, sodium cyanoborohydride or the like; sodium dithionite in alkaline medium; Borane-pyridine, Ti(OiPr)₄/NaBH₃CN, borohydride exchange resin, Zn/AcOH, NaBH₄/Mg(ClO₄)₂, and Zn(BH₄)₂/ZnCl₂ or any combination thereof; or any other suitable reduction conditions known in the art.

Step (b) of the eighth embodiment and step (a) of the ninth embodiment may be carried out in a suitable solvent. Suitable solvents that may be used based on the compatibility with the above reducing agent, which is known to a person skilled in the art include, but are not limited to: water, alcohols, such as for example, methanol, ethanol, propanol, butanol, pentanol, ethylene glycol, glycerol, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, methyl tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N,N-dimethylacetamide, N-methylpyrrolidone, pyridine, dimethylsulphoxide, sulpholane, formamide, acetamide, propanamide, or the like; nitromethane; or any mixtures thereof.

Suitable temperatures for the reaction of step (b) of the eighth embodiment and step (a) of the ninth embodiment may be less than about 200° C., less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

Step (b) of the eighth embodiment and step (a) of the ninth embodiment may optionally be carried out in an inert atmosphere such as nitrogen or argon.

The reaction mixture obtained in step (b) of the eighth embodiment and step (a) of the ninth embodiment may be optionally filtered to remove any insoluble solids, or particles may be removed by methods such as decantation, centrifugation, gravity filtration, suction filtration, or any other technique for the removal of solids.

The product of Step (b) of the eighth embodiment and step (a) of the ninth embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound is not degraded in quality, at atmospheric pressure or under a reduced pressure. The drying may be carried out for any desired times until the required purity is achieved. For example, it may vary from about 1 hour to about 10 hours, or longer.

Optionally the product of step (b) of the eighth embodiment and step (a) of the ninth embodiment may be used directly in the next step without isolation.

Step (c) of the tenth embodiment, step (b) of the eleventh embodiment and step (a) of the twelfth embodiment may be carried out in a suitable solvent. Suitable solvents that may be used include, but are not limited to: ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N, N-dimethylacetamide, N-methylpyrrolidone, pyridine, dimethylsulphoxide, sulpholane, formamide, acetamide, propanamide, or the like; nitromethane; or any mixtures thereof.

Suitable temperatures for the reaction of step (c) of the tenth embodiment, step (b) of the eleventh embodiment and step (a) of the twelfth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

The reaction mixture obtained in step (c) of the tenth embodiment, step (b) of the eleventh embodiment and step (a) of the twelfth embodiment may be optionally filtered to remove any insoluble solids, or particles may be removed by methods such as decantation, centrifugation, gravity filtration, suction filtration, or any other technique for the removal of solids.

The product of step (c) of the tenth embodiment, step (b) of the eleventh embodiment and step (a) of the twelfth embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation of compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound, at atmospheric pressure or under a reduced pressure. The drying may be carried out for any desired times until the required purity is achieved. For example, it may vary from about 1 hour to about 10 hours, or longer.

Optionally the product obtained in the step (c) of the tenth embodiment, step (b) of the eleventh embodiment and step (a) of the twelfth embodiment may be used directly in the next step without isolation.

Step (d) of the tenth embodiment, step (c) of the eleventh embodiment, step (b) of the twelfth embodiment and step (a) of the thirteenth embodiment may involve hydrolysis in the presence of base. Suitable bases that may be used include but not limited to metal alkoxides such as sodium methoxide, potassium methoxide or the like; alkali metal hydroxides, such as, for example, lithium hydroxide, sodium hydroxide, potassium hydroxide, and cesium hydroxide; alkaline earth metal hydroxides, such as, for example, barium hydroxide, strontium hydroxide, magnesium hydroxide, calcium hydroxide, or the like; alkali metal carbonates, such as, for example, sodium carbonate, potassium carbonate, lithium carbonate, cesium carbonate, or the like; alkaline earth metal carbonates, such as, for example, magnesium carbonate, calcium carbonate, or the like; alkali metal bicarbonates, such as, for example, sodium bicarbonate, potassium bicarbonate, or the like; hydrazine hydrate; anhydrous hydrazine, aqueous methyl amine; methanolic amine or mixtures thereof.

Step (d) of the tenth embodiment, step (c) of the eleventh embodiment, step (b) of the twelfth embodiment and step (a) of the thirteenth embodiment may be carried out in a suitable solvent. Suitable solvents that may be used include, but are not limited to: water; alcohols, such as for example, methanol, ethanol, propanol, butanol, pentanol, ethylene glycol, glycerol, or the like; ketones, such as for example, acetone, butanone, pentanone, methyl isobutyl ketone, or the like; esters, such as for example, ethyl formate, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N, N-dimethylacetamide, N-methylpyrrolidone, pyridine, dimethylsulphoxide, sulpholane, formamide, acetamide, propanamide, or the like; nitromethane; or any mixtures thereof.

Suitable temperatures for the reaction of step (d) of the tenth embodiment, step (c) of the eleventh embodiment, step (b) of the twelfth embodiment and step (a) of the thirteenth embodiment may be less than about 150° C., less than about 100° C., less than about 80° C., less than about 60° C., less than about 40° C., less than about 30° C., less than about 20° C., less than about 10° C., less than about 0° C., less than about −10° C., less than about −20° C., less than about −30° C., less than about −40° C. or any other suitable temperatures.

The reaction mixture obtained in step (d) of the tenth embodiment, step (c) of the eleventh embodiment, step (b) of the twelfth embodiment and step (a) of the thirteenth embodiment may be optionally filtered to remove any insoluble solids, or particles may be removed by methods such as decantation, centrifugation, gravity filtration, suction filtration, or any other technique for the removal of solids.

The product of step (d) of the tenth embodiment, step (c) of the eleventh embodiment, step (b) of the twelfth embodiment and step (a) of the thirteenth embodiment may be isolated directly from the reaction mixture itself after the reaction is complete, or after conventional work up with techniques such as filtration, quenching with a suitable reagent, extraction or the like.

Isolation of compound may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Suitable solvents that may be used for isolation of compound include, but are not limited to: ketones, such as for example, acetone, methyl isobutyl ketone or the like; esters, such as for example, methyl acetate, ethyl acetate, propyl acetate, butyl acetate, methyl propanoate, ethyl propanoate, methyl butanoate, ethyl butanoate, or the like; ethers, such as for example, diethyl ether, diisopropyl ether, t-butyl methyl ether, dibutyl ether, 1,2-dimethoxyethane, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitromethane; and any mixtures thereof.

The recovered solid may be optionally further dried. Drying may be carried out in a tray dryer, vacuum oven, air oven, fluidized bed dryer, spin flash dryer, flash dryer, or the like. The drying may be carried out at temperatures less than about 150° C., less than about 120° C., less than about 100° C., less than about 80° C., less than about 60° C., less than about 40° C. or any other suitable temperatures as long as the compound, at atmospheric pressure or under a reduced pressure. The drying may be carried out for any desired times until the required purity is achieved. For example, it may vary from about 1 hour to about 10 hours, or longer.

Optionally the product of step (d) of the tenth embodiment, step (c) of the eleventh embodiment, step (b) of the twelfth embodiment and step (a) of the thirteenth embodiment may be used directly in the next step without isolation.

Step (a) of the fourteenth embodiment may be affected in presence of reagent like CH₃MgX where X includes chloro, bromo, iodo; methyl lithium or the like or any other suitable reagent known in the art.

Step (a) of the fourteenth embodiment may be carried out in a suitable solvent. Suitable solvents that may be used include, but are not limited to: for example, ethers, such as for example, diethyl ether, diisopropyl ether, t-butyl methyl ether, dibutyl ether, tetrahydrofuran, 1,2-dimethoxyethane, 2-methoxyethanol, 2-ethoxyethanol, anisole, or the like; aliphatic or alicyclic hydrocarbons, such as for example, hexane, heptane, pentane, cyclohexane, methylcyclohexane, or the like; halogenated hydrocarbons, such as for example, dichloromethane, chloroform, 1,1,2-trichloroethane, 1,2-dichloroethene, or the like; aromatic hydrocarbons, such as for example, toluene, xylene, chlorobenzene, tetralin, or the like; nitriles, such as for example, acetonitrile, propionitrile, or the like; polar aprotic solvents, such as for example, N,N-dimethylformamide, N, N-dimethylacetamide, N-methylpyrrolidone, pyridine, dimethylsulphoxide, sulpholane, formamide, acetamide, propanamide, or the like; nitromethane; or any mixtures thereof.

Suitable temperatures for the reaction of (a) of the fourteenth embodiment may be less than about 150° C., less than about 100° C., less than about 80° C., less than about 60° C., less than about 40° C., less than about 30° C., less than about 20° C., less than about 10° C., less than about 0° C., less than about −10° C. or any other suitable temperatures.

The reaction mixture obtained in step (a) of the fourteenth embodiment may be optionally filtered to remove any insoluble solids, or particles may be removed by methods such as decantation, centrifugation, gravity filtration, suction filtration, or any other technique for the removal of solids.

The product of step (a) of the fourteenth embodiment may be isolated directly from the reaction mixture itself after the reaction is complete in step (b), or after conventional work up with techniques such as filtration, quenching with a suitable reagent, extraction or the like.

The isolation in step (a) of the fourteenth embodiment may involve methods including removal of solvent, cooling, concentrating the reaction mass, adding an anti-solvent, extraction with a solvent, or the like. Stirring or other alternate methods, such as for example, shaking, agitation, or the like, that mix the contents may also be employed for isolation.

Various compounds described in different embodiments herein above may be conveniently converted in to lorcaserin or its salts by conventional methods or methods known in the art. Further, starting from appropriate isomers or enantiomers of starting materials, wherever applicable, the processes described in different embodiments herein above may lead to corresponding isomers or enantiomers. Such processes are also contemplated in this application.

In twenty second embodiment, the present application provides an amorphous form of lorcaserin hydrochloride.

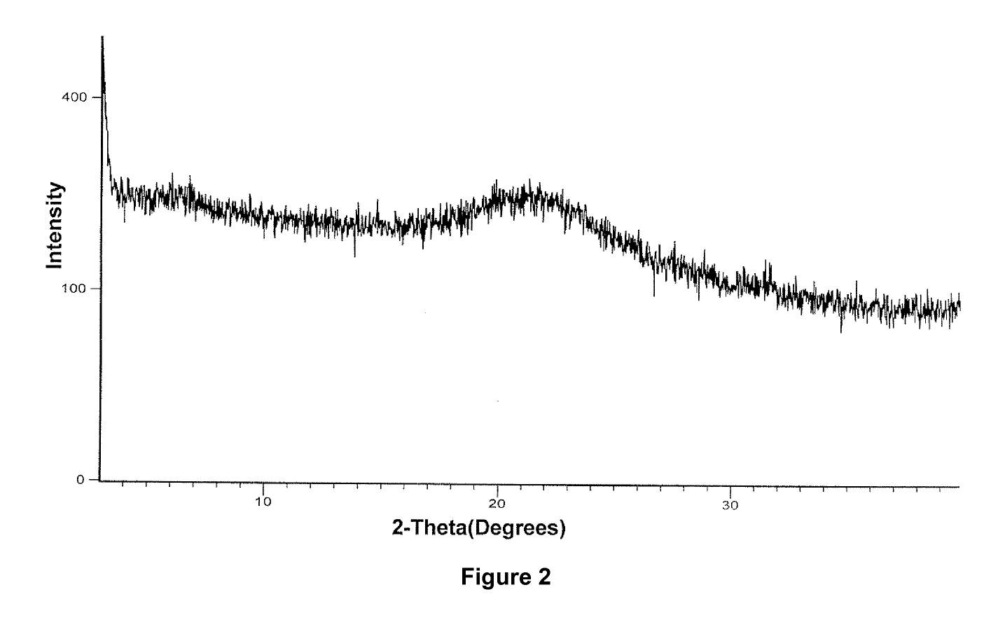

In twenty third embodiment, the present application provides an amorphous form of lorcaserin hydrochloride characterized by X-ray powder diffraction pattern substantially as illustrated by FIG. 2.

In twenty fourth embodiment, the present application provides a process for the preparation of amorphous form of lorcaserin hydrochloride, comprising:

a) providing a solution of lorcaserin hydrochloride in a suitable solvent or mixtures thereof; and

b) isolating amorphous form of lorcaserin hydrochloride.

Providing a solution of lorcaserin hydrochloride in step a) includes:
i) direct use of a reaction mixture containing lorcaserin hydrochloride that is obtained in the course of its synthesis; or
ii) dissolving lorcaserin hydrochloride in a solvent.

Any physical form of lorcaserin hydrochloride may be utilized for providing the solution of lorcaserin hydrochloride in step a). The dissolution temperatures may range from about 0° C. to about the reflux temperature of the solvent, or less than about 60° C., less than about 50° C., less than about 40° C., less than about 30° C., less than about 20° C., less than about 10° C., or any other suitable temperatures, as long as a clear solution of lorcaserin hydrochloride is obtained without affecting its quality. The solution may optionally be treated with carbon, flux-calcined diatomaceous earth (Hyflow) or any other suitable material to remove color, insoluble materials, improve clarity of the solution, and/or remove impurities adsorbable on such material. Optionally, the solution obtained above may be filtered to remove any insoluble particles. The insoluble particles may be removed suitably by filtration, centrifugation, decantation, or any other suitable techniques under pressure or under reduced pressure. The solution may be filtered by passing through paper, glass fiber, cloth or other membrane material, or a bed of a clarifying agent such as Celite® or Hyflow. Depending upon the equipment used and the concentration and temperature of the solution, the filtration apparatus may need to be preheated to avoid premature precipitation.

In embodiments, lorcaserin hydrochloride may be dissolved in any suitable solvent. Suitable solvents include any solvents that have no adverse effect on the compound and may dissolve the starting material to a useful extent. Examples of such solvents include, but are not limited to: ethers, such as diethyl ether, diisopropyl ether, tetrahydrofuran, dioxane, or dimethoxyethane; ketones, such as acetone, methyl ethyl ketone, methyl isobutyl ketone, or diethyl ketone; esters, such as ethyl acetate, propyl acetate, isopropyl acetate, or butyl acetate; alcohols, such as methanol, ethanol, 1-propanol, 2-propanol (isopropyl alcohol), 2-methoxyethanol, 1-butanol, 2-butanol, isobutyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, cyclohexanol, glycerol, or C1-C6 alcohols; nitriles, such as acetonitrile or propionitrile; amides, such as formamide, N,N-dimethylformamide, N,N-dimethylacetamide, N-methyl-2-pyrrolidone, or hexamethyl phosphoric triamide; sulfoxides, such as dimethylsulfoxide; halogenated hydrocarbons, such as dichloromethane, chloroform, carbon tetrachloride, or chlorobenzene; aromatic hydrocarbons, such as toluene; or any mixtures of two or more thereof.

Step b) involves isolating amorphous form of lorcaserin hydrochloride from the solution obtained in step a). Isolation of amorphous form of lorcaserin hydrochloride in step b) may involve methods including removal of solvent, crash cooling, flash evaporation, rotational drying, spray drying, thin-film drying, agitated nutsche filter drying or any other suitable technique. The amorphous form of lorcaserin hydrochloride as isolated may carry some amount of occluded mother liquor and may have higher than desired levels of impurities. If desired, this amorphous form may be washed with a solvent or a mixture of solvents to wash out the impurities.

Suitable temperatures for isolation may be less than about 120° C., less than about 80° C., less than about 60° C., less than about 40° C., less than about 30° C., less than about 20° C., less than about 10° C., less than about 0° C., less than about −10° C., less than about −40° C. or any other suitable temperatures.

The recovered solid may optionally be dried. Drying may be carried out in a tray dryer, vacuum oven, cone vacuum dryer, rotary vacuum dryer, fluidized bed dryer, spin flash dryer, flash dryer, or any other suitable dryer. The drying may be carried out at temperatures less than about 100° C., less than about 80° C., less than about 60° C., less than about 50° C., less than about 30° C., or any other suitable temperatures, at atmospheric pressure or under a reduced pressure, as long as the lorcaserin hydrochloride is not degraded in its quality. Optionally drying may be carried out using an inert gas such as nitrogen or argon. The drying may be carried out for any desired times until the required product quality is achieved. The dried product may optionally be subjected to a size reduction procedure to produce desired particle sizes. Milling or micronization may be performed before drying, or after the completion of drying of the product. Techniques that may be used for particle size reduction include, without limitation, ball, roller or hammer milling; or jet milling.

In twenty fifth embodiment, the present application provides pharmaceutical composition comprising amorphous form of lorcaserin hydrochloride and one or more pharmaceutically acceptable carriers.

In twenty sixth embodiment, the present application provides amorphous solid dispersion comprising lorcaserin hydrochloride and one or more pharmaceutically acceptable carriers.

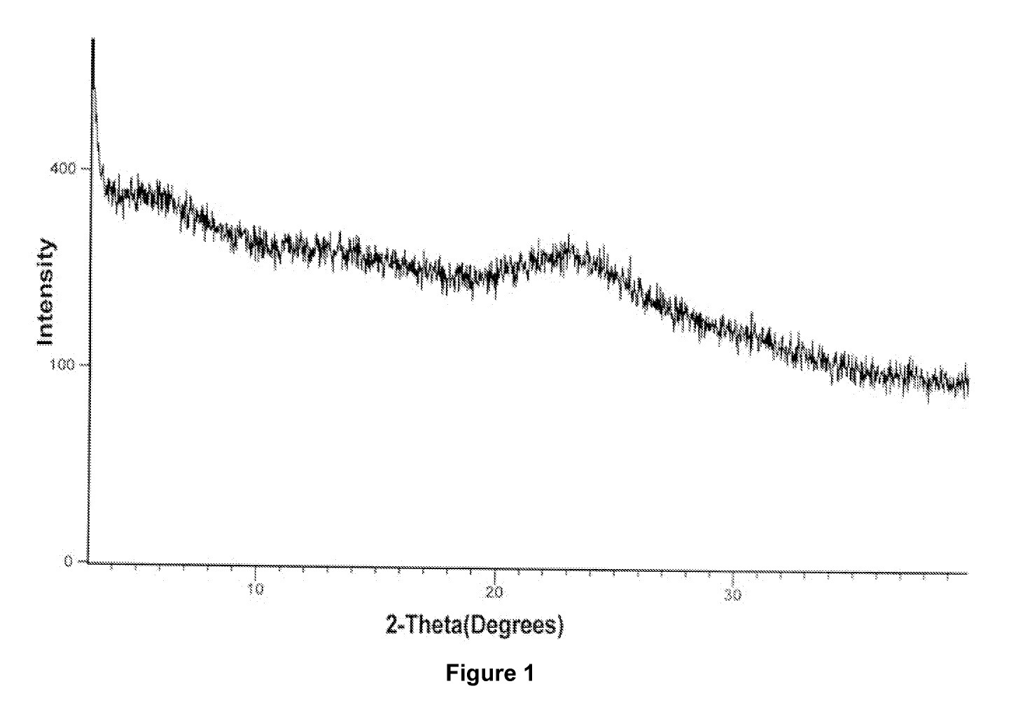

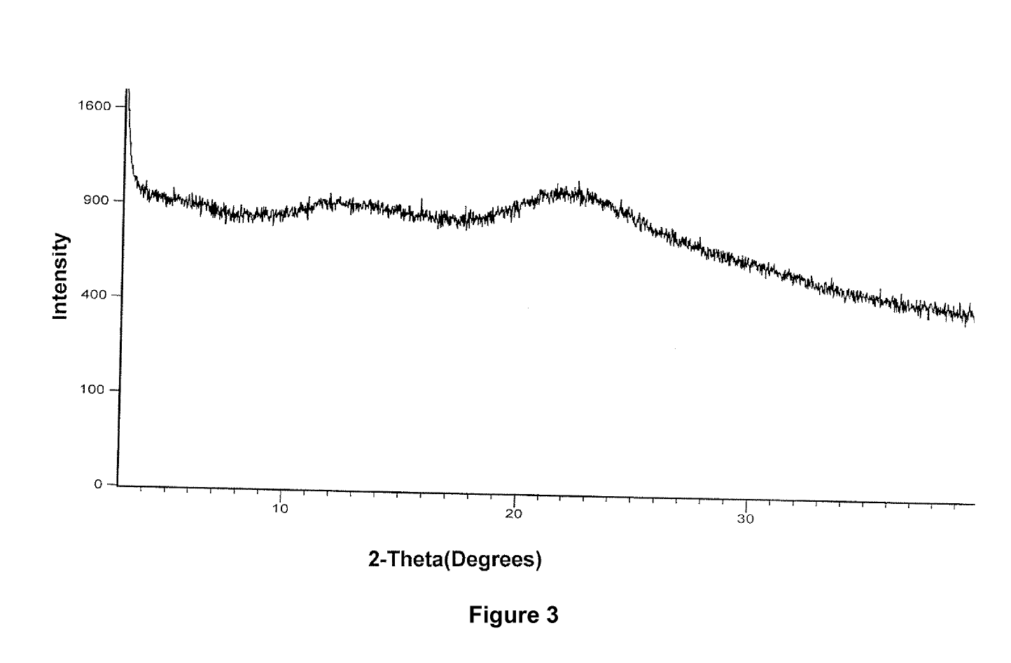

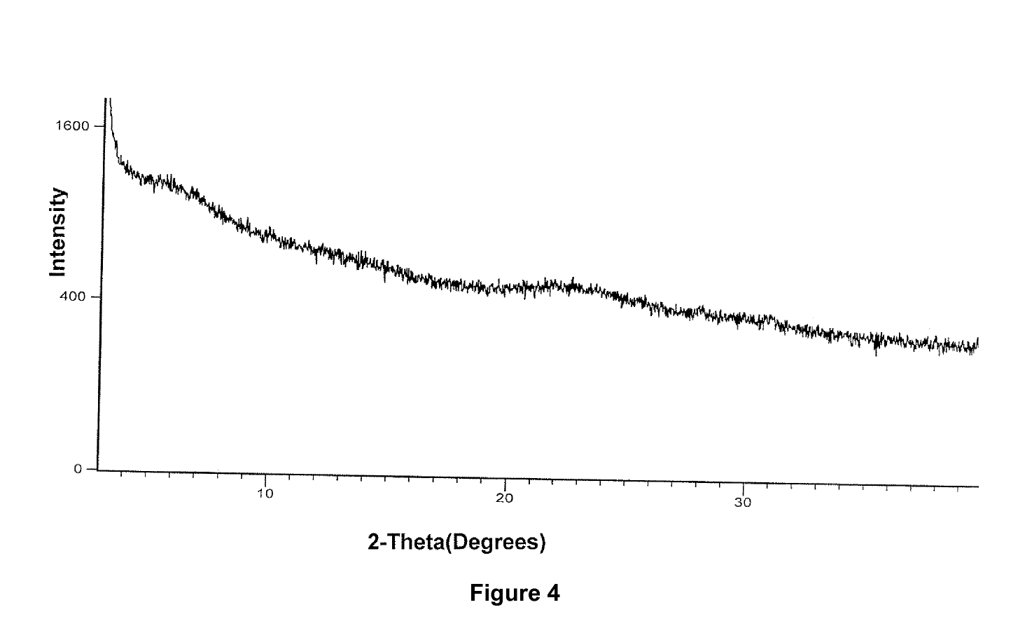

In twenty seventh embodiment, the present application provides an amorphous solid dispersion comprising lorcaserin hydrochloride and one or more pharmaceutically acceptable carriers characterized by X-ray powder diffraction pattern substantially as illustrated by FIG. 1, FIG. 3 or FIG. 4. Pharmaceutically acceptable carriers include, but are not limited to, mannitol, lactose, fructose, sorbitol, xylitol, maltodextrin, dextrates, dextrins, lactitol, inositol, trehalose, maltose, raffinose, α-, β- and γ-cyclodextrins, gum arabic, sodium alginate, propylene glycol alginate, agar, gelatin, tragacanth, xanthan gum, starch, lectins, urea, chitosan, chitosan glutamate, hydroxypropyl β-cyclodextrin chitosan, hydroxypropylmethylcellulose (HPMC), hydroxypropylcellulose (HPC), methylcellulose (MC), cellulose acetate phthalate (CAP), hydroxypropylmethylcellulose phthalate (HPMC-P), hydroxylpropyl methylcellulose acetate succinate (HPMC-AS), carboxymethylethylcellulose (CMEC), carboxymethyl cellulose, sodium carboxymethyl cellulose, cellulose acetate butyrate, hydroxyethyl cellulose, ethyl cellulose, co-(lactic/glycolic)copolymers, poly(orthoester), polyvinyl chloride, polyvinyl acetate, ethylene vinyl acetate, carbopols, silicon elastomers, polyacrylic polymers, polyvinylacetal diethylaminoacetate, aminoalkyl methacrylate copolymer E, aminoalkyl methacryl copolymer RS, methacrylic acid copolymer L, methacrylic acid copolymer LD, methacrylic acid copolymer S, and carboxylvinyl polymer, polyvinylpyrrolidones (homopolymers or copolymers of N-vinyl pyrrolidone), polyethyleneglycols of various molecular weights, polyethylene-/polypropylene-/polyethylene-oxide block copolymers, polymethacrylates, polyvinylalcohol (PVA) and co-polymers thereof with PVP or with other polymers, polyacrylates, hypromellose phthalates, polyhydric alcohols, polyethylene glycols, polyethylene oxides, polyoxyethylene derivatives, organic amines such as alkyl amines (primary, secondary, and tertiary), aromatic amines, alicyclic amines, cyclic amines, aralkyl amines, hydroxylamine or its derivatives, hydrazine or its derivatives, and guanidine or its derivatives; diluents such as starches and derivative thereof, e.g. dextrin, pullulan, corn starch and potato starch pregelatinized starches; lactose, sucrose, glucose, reduced maltose, mannitol, sorbitol, xylitol, trehalose, powdered cellulose, microcrystalline cellulose, dicalcium phosphate, tricalcium phosphate, crystalline cellulose/carmellose sodium, hydroxypropyl cellulose, magnesium aluminometasilicate, silicon dioxide, light anhydrous silicic acid or the like; binders such as acacia, guar gum, tragacanth, gelatin, polyvinylpyrrolidones, hydroxypropyl celluloses, hydroxypropyl methylcelluloses, pregelatinized starches or the like; disintegrants such as hydroxypropyl cellulose, low-substituted hydroxypropyl cellulose, croscarmellose sodium, a starch, methylcellulose, sodium alginate, sodium carboxymethyl starch, carmellose calcium, carmellose sodium, crystalline cellulose and crystalline cellulose/carmellose sodium, sodium starch glycolate, pregelatinized starches, crospovidones, colloidal silicon dioxide or the like; lubricants such as stearic acid, magnesium stearate, talc, light anhydrous silicic acid, calcium stearate, zinc stearate, magnesium oxide, sodium lauryl sulfate, sodium stearyl fumarate, magnesium aluminometasilicate or the like; flavoring agents such as sucrose, aspartame, mannitol, dextran, saccharin, menthol, citric acid, tartaric acid, malic acid, ascorbic acid, sweet hydrangea leaves, fennel, ethanol, fructose, xylitol, glycyrrhizinic acid, purified sucrose, L-glutamine, cyclodextrin, peppermint, methyl salicylate or the like; surfactants such as sodium lauryl sulfate, polysolvate 80, sucrose fatty acid ester, polyoxyl 40 stearate, polyoxyethylene 60 hydrogenated castor oil, sorbitan monostearate, sorbitan monopalmitate or the like; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, waxes or the like. Other pharmaceutically acceptable carriers that can be used include, but are not limited to, film formers, plasticizers, colorants, viscosity enhancers, preservatives, antioxidants, or the like.

The use of mixtures of more than one of the pharmaceutical acceptable carriers to provide desired release profiles or for the enhancement of stability is within the scope of this invention. Also, all viscosity grades, molecular weights, commercially available products, their copolymers, and mixtures are all within the scope of this invention without limitation.

Any suitable quantity of pharmaceutical acceptable carriers can be used for the preparation of the said amorphous solid dispersion. For example, about 0.1, about 0.5, about 1.0, about 2.0, about 3.0, about 4.0, about 5.0, about 6.0, about 7.0, about 8.0, about 9.0 or about 10.0 portions by weight of pharmaceutical acceptable carriers can be used per one portion by weight of lorcaserin hydrochloride.

In twenty eighth embodiment, the present application provides a process for preparing an amorphous solid dispersion comprising lorcaserin hydrochloride and one or more pharmaceutically acceptable carriers, comprising the steps of:
a) dissolving or dispersing lorcaserin hydrochloride and one or more pharmaceutically acceptable carriers in a suitable solvent or mixtures thereof; and
b) isolating an amorphous solid dispersion comprising lorcaserin hydrochloride and one or more pharmaceutically acceptable carriers.

Lorcaserin hydrochloride used in step-a includes, direct use of a reaction mixture containing lorcaserin hydrochloride obtained in the course of its manufacture, if desired, after addition of one or more pharmaceutically acceptable carriers. Optionally, lorcaserin hydrochloride can be dissolved or dispersed in a suitable solvent or mixture of solvent, either alone followed by addition of one or more pharmaceutically acceptable carriers, or in combination with one or more pharmaceutically acceptable carriers. Optionally, auxiliaries such as diluents or disintegrant can be added during dissolution or dispersion.

Any physical form of lorcaserin hydrochloride, such as crystalline, amorphous or their mixtures can be utilized in step a.

The dissolution temperatures can range from about 0° C. to about the reflux temperature of the solvent, less than about 100° C., less than about 90° C., less than about 80° C., less than about 70° C., less than about 60° C., less than about 50° C., less than about 40° C., less than about 30° C., less than about 20° C., less than about 10° C., or any other suitable temperatures, as long as a clear solution of lorcaserin hydrochloride is obtained without affecting its quality. The solution can optionally be treated with carbon, flux-calcined diatomaceous earth (Hyflow) or any other suitable material to remove color, insoluble materials, improve clarity of the solution, and/or remove impurities adsorbable on such material. Optionally, the solution obtained above can be filtered to remove any insoluble particles. The insoluble particles can be removed suitably by filtration, centrifugation, decantation, or any other suitable techniques under pressure or under reduced pressure. The solution can be filtered by passing through paper, glass fiber, cloth or other membrane material, or a bed of a clarifying agent such as Celite® or Hyflow. Depending upon the equipment used and the concentration and temperature of the solution, the filtration apparatus can need to be preheated to avoid premature precipitation.

When the solution or dispersion of lorcaserin hydrochloride is prepared together with a pharmaceutically acceptable carrier, the order of charging different materials to the solution is not critical for obtaining the desired solid dispersion. A specific order may be preferred with respect to the equipment being used and will be easily determined by a person skilled in the art. Lorcaserin hydrochloride or pharmaceutically acceptable carrier may be completely soluble in the solvent or they may form dispersion. In embodiments, lorcaserin hydrochloride and the pharmaceutically acceptable carrier may be separately dissolved either in the same solvent or in different solvents, and then combined to form a mixture.

Suitable solvents that can be used in step a) include, but are not limited to, water; alcohol; ketone; halogenated hydrocarbon; ester; ether; nitrile; polar aprotic; or mixtures thereof.

Isolation of an amorphous solid dispersion comprising lorcaserin hydrochloride and one or more pharmaceutically acceptable carriers in step b) can involve methods including removal of solvent, crash cooling, flash evaporation, rotational drying, spray drying, thin-film drying, agitated nutsche filter drying, pressure nutsche filter drying, freeze-drying or any other suitable technique. An amorphous solid dispersion as isolated can carry some amount of occluded mother liquor and may have higher than desired levels of impurities. If desired, amorphous solid dispersion may be washed with a solvent or a mixture of solvents to wash out the impurities.

Suitable temperatures for isolation may be less than about 120° C., less than about 80° C., less than about 60° C., less than about 40° C., less than about 30° C., less than about 20° C., less than about 10° C., less than about 0° C., less than about −10° C., less than about −40° C. or any other suitable temperatures.

Isolation may be carried out by freeze drying (lyophilization) the solution at low temperatures and reducing the pressure to remove the solvent from the frozen solution. Temperatures that may be required to freeze the solution, depending on the solvent chosen to make the solution, may range from about −80° C. to about 20° C. Temperatures that may be required to remove the solvent from the frozen solution may be less than about 20° C., less than about 0° C., less than about −20° C., less than about −40° C., less than about −60° C., less than about −80° C., or any other suitable temperatures.

Optionally, isolation may be effected by combining a suitable anti-solvent with the solution obtained in step a). Anti-solvent as used herein refers to a liquid in which lorcaserin hydrochloride is less soluble or poorly soluble. An inert anti-solvent has no adverse effect on the reaction and it may assist in the solidification or precipitation of the dissolved starting material. Suitable anti-solvents that may be used include, but are not limited to: saturated or unsaturated, linear or branched, cyclic or acyclic, C₁ to C₁₀ hydrocarbons, such as heptanes, cyclohexane, or methylcyclohexane; water; or any mixtures thereof.

The recovered solid may optionally be dried. Drying may be carried out in a tray dryer, vacuum oven, air oven, cone vacuum dryer, rotary vacuum dryer, fluidized bed dryer, spin flash dryer, flash dryer, or the like. The drying may be carried out at temperatures less than about 100° C., less than about 80° C., less than about 60° C., less than about 50° C., less than about 30° C., or any other suitable temperatures, at atmospheric pressure or under a reduced pressure, as long as the lorcaserin hydrochloride is not degraded in quality. The drying may be carried out for any desired times until the required product quality is achieved. The dried product may optionally be subjected to a size reduction procedure to produce desired particle sizes. Milling or micronization may be performed before drying, or after the completion of drying of the product. Techniques that may be used for particle size reduction include, without limitation, ball, roller or hammer milling; or jet milling.

In twenty ninth embodiment, the present application provides a process for preparing an amorphous solid dispersion comprising lorcaserin hydrochloride and one or more pharmaceutically acceptable carriers, comprising the steps of:
(a) dissolving or dispersing lorcaserin hydrochloride under heating in one or more pharmaceutically acceptable carriers; and
(b) cooling down the mixture.

The methods for dissolving or dispersing lorcaserin hydrochloride include stirring by heating up to or higher than the melting point or the softening point of lorcaserin hydrochloride or pharmaceutically acceptable carrier(s). Optionally, pressure can be applied using kneading machine along with heating. Plasticizers such as, polyethyleneglycol, sucrose fatty acid ester, glycerine fatty acid ester, propylene glycol, triethyl citrate, castor oil and triacetin; and surfactants, e.g. sodium lauryl sulfate, polysolvate 80, sucrose fatty acid ester, polyoxyl 40 stearate, polyoxyethylene 60 hydrogenated castor oil, sorbitan monostearate, and sorbitan monopalmitate can be added as additives.

The amorphous solid dispersion by the melt method can be produced using agitation granulators with heating, for example. More concretely, a mixture of lorcaserin hydrochloride and one or more pharmaceutically acceptable carriers can be prepared in advance. The above plasticizers or surfactants may be added to the mixture, if necessary. The conditions such as the treatment temperature and time vary depending on pharmaceutical carrier(s), additives and the like. The treatment temperature can be within the range of room temperature to about 300° C.; and the treatment time can be within the range of a few minutes to several hours. The cooling temperature can be within the range of about −100° C. to room temperature.

In thirtieth embodiment, the present application provides a process for preparing an amorphous solid dispersion comprising lorcaserin hydrochloride and one or more pharmaceutically acceptable carriers, comprising the steps of:
(c) dissolving or dispersing lorcaserin hydrochloride and one or more pharmaceutically acceptable carriers in a suitable solvent or mixture of solvents;
(d) spray drying the mixture to obtain the said amorphous solid dispersion.

Suitable solvents that can be used during spray drying include, but are not limited to, water; alcohol; ketone; halogenated hydrocarbon; ester; ether; nitrile; polar aprotic; or mixtures thereof.

The spray drying technique involves dissolving lorcaserin hydrochloride and one or more pharmaceutically acceptable excipients in suitable solvent or mixtures thereof. The solution may be spray dried at 40 to 100° C. inlet temperature, at a feed rate of 3 ml/min. to 15 ml/minute and at 4.5 to 6.5 kg/cm² atomization pressure.

In thirty first embodiment, the present application provides pharmaceutical composition comprising amorphous solid dispersion of lorcaserin hydrochloride and pharmaceutically acceptable carriers.

In an embodiment, the present application provides pharmaceutical composition comprising amorphous form of lorcaserin hydrochloride and pharmaceutically acceptable carriers.

Amorphous form of lorcaserin hydrochloride or amorphous solid dispersion comprising lorcaserin hydrochloride may be further formulated as: solid oral dosage forms such as, but not limited to: powders, granules, pellets, tablets, and capsules; liquid oral dosage forms such as but not limited to syrups, suspensions, dispersions, and emulsions; and injectable preparations such as but not limited to solutions, dispersions, and freeze dried compositions. Formulations may be in the forms of immediate release, delayed release or modified release. Further, immediate release compositions may be conventional, dispersible, chewable, mouth dissolving, or flash melt preparations, and modified release compositions that may comprise hydrophilic or hydrophobic, or combinations of hydrophilic and hydrophobic, release rate controlling substances to form matrix or reservoir or combination of matrix and reservoir systems. The formulation may be prepared using techniques such as direct blending, dry granulation, wet granulation, and extrusion and spheronization. Formulation may be presented as uncoated, film coated, sugar coated, powder coated, enteric coated, and modified release coated.

Certain specific aspects and embodiments of the present invention will be explained in more detail with reference to the following examples, which are provided for purposes of illustration and should not be construed as limiting the scope of the present invention in any manner.

All PXRD data reported herein are obtained using a Bruker AXS D8 Advance Powder X-ray Diffractometer or a PANalytical X-ray Diffractometer, using copper Kα radiation wavelength 1.5418 Å.

### EXAMPLES

### Example 1

### Preparation of Methyl-2-(3-Chlorophenyl) Acetate

3-chloro phenyl acetic acid (10.0 g) was added to a mixture containing methanol (50 mL) and sulfuric acid (2.0 mL) at 25-35° C. The mixture was heated to reflux temperature. The refluxing was continued till substantial completion of the reaction. The solvent in the reaction mass was distilled under reduced pressure at 55° C. Water (50 mL) was added and the pH of the mixture was adjusted to pH 8 with 20% sodium carbonate solution (50.0 mL). Ethyl acetate (100 mL) was charged and aqueous and organic layer were separated. The aqueous layer was extracted with ethyl acetate (50 mL). The ethyl acetate layers were combined and washed with water (50 mL). The organic layer was distilled at 55° C. under reduced pressure to obtain the title compound.

Yield: 10.3 g.

### Example 2

### Preparation of Methyl-2-(3-chlorophenyl) propanoate

Sodium hydride (60% in oil) (1.1 g) was added to a pre-cooled (−20° C.) THF (30 mL) under nitrogen atmosphere. Methyl-2-(3-chlorophenyl) acetate (5.0 g) was slowly added at −20 to −10° C. for 10 minutes and the resulting mixture was stirred for 45 minutes at −18 to −15° C. Methyl iodide (1.7 mL) dissolved in THF (10 mL) was added slowly at −20° C. and maintained the reaction mixture till substantial completion of the reaction. Ammonium chloride solution (50 mL) was added to the reaction mass and stirred for 10 minutes at 0° C. Ethyl acetate (50 mL) was charged and separated the aqueous and organic layer. The aqueous layer was extracted with ethyl acetate (50 mL). The ethyl acetate layers were combined and washed with water (30 mL). The resulting organic layer was distilled under reduced pressure at 58° C. to afford the title compound.

Yield: 5.1 g.

### Example 3

### Preparation of 2-(3-chlorophenyl) propan-1-ol

Methyl-2-(3-chlorophenyl) propanoate (5.0 g) was added to THF (20 mL) and cooled to 10° C. NaBH₄ (2.8 g) was added slowly during 15 minutes at 10° C. and the resulting mixture was stirred at 25° C. for 20 minutes. Methanol (5.0 mL) was added slowly at 25-35° C. and maintained the reaction mixture till substantial completion of reaction. The reaction mixture was cooled to 10° C. and ammonium chloride solution (50 mL) was added and stirred for 30 minutes. Ethyl acetate (50 mL) and Water (30 mL) was charged at 0-10° C. and extracted the compound from ethyl acetate (2×50 mL). Organic layer was washed with water (30 mL) and the solvent was distilled under reduced pressure at 55° C. to afford the title compound.

Yield: 4.1 g.

### Example 4

### Preparation of 2-(3-chlorophenyl) propyl methanesulfonate

2-(3-chlorophenyl) propan-1-ol (15.0 g), DCM (150 mL) and triethylamine (25 mL) were cooled to 0-5° C. Methane sulphonyl chloride (8.6 mL) was added at 3° C. for 30 minutes and the reaction mass was stirred at 28° C. till substantial completion of the reaction. Water (100 mL) was added slowly to the reaction mass at 28° C. and stirred for 10 minutes. Organic and aqueous layer were separated. Aqueous layer was extracted with dichloromethane (100 mL). The combined organic layer was washed with water (100 mL) and the organic layer was distilled under reduced pressure at 46° C. to afford the title compound.

Yield: 16.5 g.

### Example 5

### Preparation of 2-(3-chlorophenyl)-N-(2,2-dimethoxyethyl)propan-1-amine

2-(3-chlorophenyl)propylmethanesulfonate (15.0 g), 2,2-dimethoxyethan-1-amine (7.0 g) and triethylamine (30 mL) were charged into a round bottom flask and heated to reflux till substantial completion of the reaction. The solvent was distilled under reduced pressure at 65° C. to afford the title compound.

Yield: 16.0 g.

### Example 6

### Preparation of 2-(3-chlorophenyl) propyl phthalimide

2-(3-chlorophenyl) propyl methanesulfonate (1.0 g), potassium phthalimide (0.86 g) and dimethyl formamide (15.0 mL) were charged into the round bottom flask at 25-35° C. The contents were heated to 105° C. The reaction mixture was maintained at 105° C. till substantial completion of the reaction. The reaction mixture was cooled to 25-35° C. Water (30 mL) was charged to the reaction mass at 25-35° C. and the compound was extracted with ethylacetate (2×30 mL). The organic layer was washed with water (20 mL) and the solvent was distilled under reduced pressure at 60° C. to afford the title compound.

Yield: 1.4 g

### Example 7

### Preparation of 2-(3-chlorophenyl) propan-1-amine

2-(3-chlorophenyl)propylphthalimide (1.0 g), methanol (10 mL) and hydrazine hydrate (0.8 mL) were charged into the round bottom flask at 28° C. The contents were stirred and heated to reflux till substantial completion of the reaction. The reaction mass was cooled to 45° C. and distilled under reduced pressure at 45° C. Water (30 ml) and ethyl acetate (30 mL) were charged to the distilled mass at 25-30° C. and stirred. The organic and aqueous layers were separated and aqueous layer was extracted with ethyl acetate (30 mL). The combined organic layer was washed with water (20 mL). The solvent from the organic layer was distilled under reduced pressure at 45° C. to afford the title compound.

Yield: 0.3 g.

### Example 8

### Preparation of 2-(3-chlorophenyl) propanal

2-(3-chlorophenyl) propan-1-ol (1.0 g), DCM (10 mL) and 1,1,1-Triacetoxy-1,1-dihydro-1,2-benziodoxol-3(1H)-one (4.0 g) were charged into a reactor at 25-35° C. and stirred till substantial completion of the reaction. The reaction mass was filtered under reduced pressure and washed with DCM (10 mL). The solvent from the filtrate was distilled under reduced pressure below 40° C. to afford the title compound.

Yield: 1.0 g

### Example 9

### Preparation of 2-(3-chlorophenyl) propanoic acid

Methyl-2-(3-chlorophenyl) propanoate (10 g), ethanol (50 ml) and sodium hydroxide (2.0 g) were charged into a round bottom flask and heated to reflux till substantial completion of the reaction. The reaction mass was distilled under reduced pressure and water (50 mL) was charged to the distilled mass. The pH was adjusted to 1-2 with 20% aqueous HCl (10 ml). Ethyl acetate (50 mL) was charged and stirred at 28° C. The aqueous and organic layers were separated. The aqueous layer was extracted with ethyl acetate (50 ml). The combined organic layer was washed with water (30 mL). The organic layer was distilled under reduced pressure at 60° C. to afford the title compound.

Yield: 9.1 g

### Example 10

### Preparation of 2-(3-chlorophenyl) propanamide

2-(3-chlorophenyl) propanoic acid (5.0 g) and THF (50 mL) were charged into a round bottom flask at 25-35° C. and cooled to −20° C. Disopropyl ethylamine (7.0 g) was charged into a round bottom flask at −20° C. Methansulfonyl chloride (4.65 g) was added at −20 to −30° C. for 30 minutes and the reaction was maintained at −20° C. till substantial completion of the reaction. Ammonia gas was purged into the reactor and reaction was maintained at 0 to 5° C. till the pH becomes 8-10. The reaction mass was distilled under reduced pressure at 40° C. Water (50 mL) and ethyl acetate (50 mL) were charged to the distilled mass and stirred for 10 minutes. The aqueous and organic layers were separated. The aqueous layer was extracted with ethyl acetate (50 ml). The combined organic layer was distilled under reduced pressure at 38° C. Hexane (250 mL) was added to the distilled mass and stirred for 20 minutes at 30° C. The solid was filtered and washed with hexane (10 mL). The compound was dried under reduced pressure at 30° C. for 45 minutes to afford the title compound.

Yield: 2.8 g

### Example 11

### Preparation of 2-(3-chlorophenyl) propanenitrile

Sodamide (1.28 g) and THF (15 mL) were charged into a round bottom flask at 25-30° C. and cooled to 0° C. 2-(3-chlorophenyl) acetonitrile (5.0 g) dissolved in THF (10 mL) was added slowly for 20 minutes and stirred for 40 minutes at 2° C. Methyl iodide (4.68 g) was added slowly for 20 minutes at 3° C. The reaction was maintained at 0-5° C. till substantial completion of the reaction. The reaction mass was distilled under reduced pressure below 58° C. Water (20 mL) and ethyl acetate (25 mL) were added to the distilled mass and stirred for 20 minutes. The aqueous and organic layers were separated. The aqueous layer was extracted with ethyl acetate (25 ml). The combined organic layer was distilled under reduced pressure at 50° C. afford the title compound.

Yield: 5.5 g

### Example 12

### Preparation of 2-(3-chlorophenyl) propan-1-amine

2-(3-chlorophenyl) propanal (1.0 g) and ammonium formate (2.0 g) were charged into a round bottom flask and heated to 170° C. till substantial completion of the reaction. The reaction mass was cooled to 28° C. and THF (8 mL) was charged. NaBH₄ (0.5 g) was slowly added to the reaction mass and stirred till substantial completion of the reaction. The reaction mass was cooled to 0° C. Water (2 mL) was charged at 0° C. and the aqueous layer was extracted with ethylacetate (2×25 mL) at 15° C. The combined organic layer was distilled under reduced pressure at 50° C. to afford the title compound. Yield: 0.3 g

### Example 13

### Preparation of 2-(3-chlorophenyl) propan-1-amine

2-(3-chlorophenyl) propanamide (1.5 g) and THF (10 mL) were charged into a round bottom flask at 28° C. and stirred for 10 minutes. NaBH₄ (1.0 g) was charged and heated to 73° C. BF₃.Et₂O (7 mL) dissolved in THF (7.0 mL) was added slowly to reaction mass for 2 hours and maintained the reaction till substantial completion of the reaction. The reaction mass was distilled under reduced pressure at 48° C. 5% HCl solution (5 mL) was added to the distilled mass and stirred at 30° C. for 60 minutes. The contents were cooled to 20° C. and sodium bicarbonate solution (10 mL) was added. Stirred for 20 minutes and ethyl acetate (150 mL) was added. The aqueous and organic layers were separated. The aqueous layer was extracted with ethyl acetate (150 ml). The combined organic layer was distilled under reduced pressure at 25° C. afford the title compound.

Yield: 1.0 g

### Example 14

### Preparation of 1-Chloro-3-(2-nitroethenyl) benzene

3-chlorobenzaldehyde (28.1 g), glacial acetic acid (230 mL) and ammonium acetate (15.4 g) were charged into a round bottom flask and heated to reflux till substantial completion of the reaction. The reaction mass was cooled to 55° C. and distilled at 60° C. under reduced pressure. The distilled mass was cooled to 28° C. Water (280 mL) was added to the distilled mass. The aqueous layer was extracted with ethylacetate (2×90 mL). The combined organic layer was distilled under reduced pressure at 48° C. to afford the title compound. The compound was further purified by column chromatography.

Yield: 4.0 g

### Example 15

### Preparation of 1-chloro-3-(1-nitropropan-2-yl) benzene

1-Chloro-3-(2-nitroethenyl) benzene (2.0 g) and THF (20 mL) were charged into a round bottom flask and cooled to −20 to −25° C. CH₃MgBr (10 mL) dissolved in THF (10 mL) was added slowly for 15 minutes. The reaction mixture was maintained for 70 minutes at −20° C. 10% aqueous HCl (10 mL) was added to the reaction mixture at −18° C. and maintained for 60 minutes. The aqueous layer was extracted with DCM (2×40 mL) at 25° C. The combined organic layer was distilled under reduced pressure at 40° C. to afford the title compound. The compound was further purified by column chromatography.

Yield: 0.8 g

### Example 16

### Preparation of 2-(3-chlorophenyl) propan-1-amine

AlCl₃ (0.46 g) was added to the pre-cooled (5° C.) THF (15 mL) for 10 minutes and stirred for 40 minutes in a round bottom flask. THF (15 mL) and LiAlH₄ (1.63 g) were charged into a separate round bottom flask under inert atmosphere and cooled to 0-5° C. AlCl₃ mixture prepared above was added to the LiAlH₄ mixture. 2-(3-chlorophenyl) propanenitrile (2.0 g) was added to the above mixture for 20 minutes at 2° C. and stirred at 25° C. till substantial completion of the reaction. The reaction mixture was cooled to 0-5° C. and water (1.2 mL) was added for 12 minutes. 20% NaOH solution (8 mL) was added at 4° C. and stirred the reaction mass for 19 hours at 15-26° C. The reaction mass was filtered and washed with THF (15 mL). The filtrate was distilled under reduced pressure at 50° C. Ethyl acetate (20 mL) was charged and stirred the mixture for 15 minutes. Layers were separated. The aqueous layer was extracted with ethyl acetate (20 ml). The combined organic layer was dried over anhydrous sodium sulphate and distilled under reduced pressure at 50° C. to afford the title compound.

Yield: 1.2 g

### Example 17

### Preparation of 2-(3-chlorophenyl) propan-1-amine

Raney nickel (0.25 g) and ethanol (5 mL) were charged into round bottom flask at 26° C. NaBH₄ (0.5 g) was added slowly to the round bottom flask. 2-(3-chlorophenyl) propanenitrile (0.5 g) dissolved in ethanol (5 mL) was added to the above mixture for 10 minutes 26° C. The reaction mixture was maintained till substantial completion of the reaction. The reaction mass was filtered and washed with ethanol (5 mL). The filtrate was distilled under reduced pressure at 50° C. Water (5 ml) and ethyl acetate (5 mL) were charged to the distilled mass. The organic layer was separated and distilled under reduced pressure at 50° C. to afford the title compound. Yield: 0.25 g

### Example 18

### Preparation of 2-(3-chlorophenyl) propan-1-amine

2-(3-chlorophenyl) propanenitrile (1.0 g) dissolved in methanol (10 mL) was passed through H₂-Raney Nickel cartridge at 50° C. and 20 bar pressure. The reaction mass was collected in a separate reactor. The reaction mass was distilled under reduced pressure at 26° C. to afford the title compound. Yield: 0.8 g

### Example 19

### Preparation of Amorphous Solid Dispersion Comprising lorcaserin hydrochloride and PVP-k-30

Lorcaserin hydrochloride (1.0 g) and PVP-K-30 (1.0 g) were taken in methanol (50 ml) and stirred at room temperature for 15 minutes to obtain a clear solution. The resulting solution was subjected to distillation under reduced pressure at 70 to 80° C. for 30-45 minutes and further dried at 90-95° C. for 1 hour to obtain amorphous solid dispersion comprising lorcaserin hydrochloride.

### Example 20

### Preparation of Amorphous Solid Dispersion Comprising lorcaserin hydrochloride and PVP-k-30

Lorcaserin hydrochloride (1.0 g) and PVP-K-30 (1.0 g) were taken in dichloromethane (25 ml) and stirred at room temperature for 10 minutes to obtain a clear solution. The resulting solution was subjected to distillation under reduced pressure at 50 to 70° C. for 15-20 minutes and further dried at 90-95° C. for 1 hour. Material was unloaded and dried in air tray drier at 90° C. for 2 to 3 hours to obtain amorphous solid dispersion comprising lorcaserin hydrochloride.

### Example 21

### Preparation of Amorphous Solid Dispersion Comprising Lorcaserin hydrochloride and PVP-k-30

Lorcaserin Hydrochloride (12.5 g) and PVP-K-30 (37.5 gm) was taken in methanol (400 ml) and stirred at room temperature for 15 minutes to obtain a clear solution. The solution was filtered to obtain a particle free solution. The resulting solution was spray dried at below mentioned parameters to obtain amorphous solid dispersion comprising lorcaserin hydrochloride.

Operation parameters:

### Aspirator: 80%

Feed rate: 20%
Inlet nitrogen temperature: 70° C.
Outlet temperature: 47-48° C.
Atomisation Pressure: 5.0 kg/cm²

### Example 22

### Preparation of Amorphous Solid Dispersion Comprising Lorcaserin hydrochloride and PVP-k-30

Lorcaserin hydrochloride (500 mg) and PVP-K-30 (1.5 g) were taken in methanol (30 ml) and stirred at room temperature for 10 minutes to obtain a clear solution. The resulting solution was subjected to distillation under reduced pressure at 70° C. for 30-45 minutes to obtain amorphous solid dispersion comprising lorcaserin hydrochloride.

### Example 23

### Preparation of Amorphous Solid Dispersion Comprising Lorcaserin hydrochloride and HPMC-AS

Lorcaserin hydrochloride (1.0 g) and HPMC-AS (1.0 g) were taken in methanol (100 ml) and stirred at room temperature for 15 minutes to obtain a clear solution. The resulting solution was subjected to distillation under reduced pressure at 70-80° C. for 15-20 minutes and further dried at 90-95° C. for 1 hour to obtain amorphous solid dispersion comprising lorcaserin hydrochloride.

### Example 24

### Preparation of Amorphous Solid Dispersion Comprising Lorcaserin hydrochloride and HPMC-AS

Lorcaserin hydrochloride (500 mg) and HPMC-AS (500 mg) were taken in dichloromethane (25 ml) and stirred at room temperature for 10 minutes to obtain a clear solution. The resulting solution was subjected to distillation under reduced pressure at 60-70° C. for 10-15 minutes and further dried at 80° C. for 1 hour to obtain amorphous solid dispersion comprising lorcaserin hydrochloride.

### Example 25

### Preparation of Amorphous Solid Dispersion Comprising Lorcaserin hydrochloride and HPMC-AS

Lorcaserin hydrochloride (500 mg) and HPMC-AS (1.0 g) were taken in methanol (80 ml) and stirred at room temperature for 10 minutes to obtain a clear solution. The resulting solution was subjected to distillation under reduced pressure at 70° C. for 75-80 minutes to obtain amorphous solid dispersion comprising lorcaserin hydrochloride.

### Example 26

### Preparation of Amorphous Solid Dispersion Comprising Lorcaserin hydrochloride and HPMC 3CPS

Lorcaserin hydrochloride (500 mg) and HPMC 3CPS (500 mg) were taken in dichloromethane (25 ml) and stirred at room temperature for 10 minutes to obtain a clear solution. The resulting solution was subjected to distillation under reduced pressure at 60-70° C. for 10-15 minutes and further dried at 80° C. for 1 hour to obtain amorphous solid dispersion comprising lorcaserin hydrochloride.

PXRD Pattern is shown as FIG. 1.

### Example 27

### Preparation of Amorphous Solid Dispersion Comprising Lorcaserin hydrochloride and HPMC 3CPS

Lorcaserin hydrochloride (1.0 g) and HPMC 3CPS (1.0 g) were taken in methanol (100 ml) and stirred at room temperature for 10 minutes to obtain a clear solution. The resulting solution was subjected to distillation under reduced pressure at 70-85° C. for 15-20 minutes and further dried at 95° C. for 1 hour to obtain amorphous solid dispersion comprising lorcaserin hydrochloride.

### Example 28

### Preparation of Amorphous Solid Dispersion Comprising Lorcaserin hydrochloride and HPC EF

Lorcaserin hydrochloride (1.0 g) and HPC EF (1.0 g) were taken in dichloromethane (50 ml) and stirred at room temperature for 10 minutes to obtain a clear solution. The resulting solution was subjected to distillation under reduced pressure at 60-70° C. for 10-15 minutes and further dried at 80° C. for 1 hour to obtain amorphous solid dispersion comprising lorcaserin hydrochloride.

### Example 29

### Preparation of Amorphous Solid Dispersion Comprising Lorcaserin hydrochloride and HPC EF

Lorcaserin hydrochloride (1.0 g) and HPC EF (1.0 g) were taken in methanol (50 ml) and stirred at room temperature for 10 minutes to obtain a clear solution. The resulting solution was subjected to distillation under reduced pressure at 70-80° C. for 10-15 minutes and further dried at 90° C. for 1 hour to obtain amorphous solid dispersion comprising lorcaserin hydrochloride.

### Example 30

### Preparation of Amorphous Lorcaserin Hydrochloride

Lorcaserin Hydrochloride (7.0 g) was taken in Methanol (70 ml) and stirred at room temperature to obtain a clear solution. The solution was filtered on filter paper to obtain a particle free solution. The resulting solution was spray dried at below mentioned parameters to obtain the amorphous lorcaserin hydrochloride.

Operation parameters:

### Aspirator: 70%

Feed rate: 10% (3 ml/min)
Inlet nitrogen temperature: 70° C. to 75° C.
Atomisation Pressure: 5.0 kg/cm²
PXRD Pattern is shown as FIG. 2.

### Example 31

### Preparation of Amorphous Solid Dispersion Comprising Lorcaserin Hydrochloride and HPMC

Lorcaserin hydrochloride (500 mg) and Hydroxypropyl methylcellulose (HPMC) (500 mg) were taken in Methanol (100 ml) and stirred at room temperature to obtain a clear solution. The resulting solution was subjected to distillation under reduced pressure at 60 to 65° C. for 30-45 minutes to obtain the amorphous form comprising lorcaserin hydrochloride.

PXRD Pattern is shown as FIG. 3.

### Example 32

### Preparation of Amorphous Solid Dispersion Comprising Lorcaserin hydrochloride and PVP-K-30

Lorcaserin hydrochloride (1000 mg) and PVP-K-30 (1000 mg) were taken in Methanol (30 ml) and stirred at room temperature to obtain a clear solution. The resulting solution was subjected to distillation under reduced pressure at 60 to 65° C. for 30-45 minutes to obtain amorphous form comprising lorcaserin hydrochloride. PXRD Pattern is shown as FIG. 4.

### Example 33

### Preparation of Amorphous Solid Dispersion Comprising Lorcaserin hydrochloride and PVP-K-30

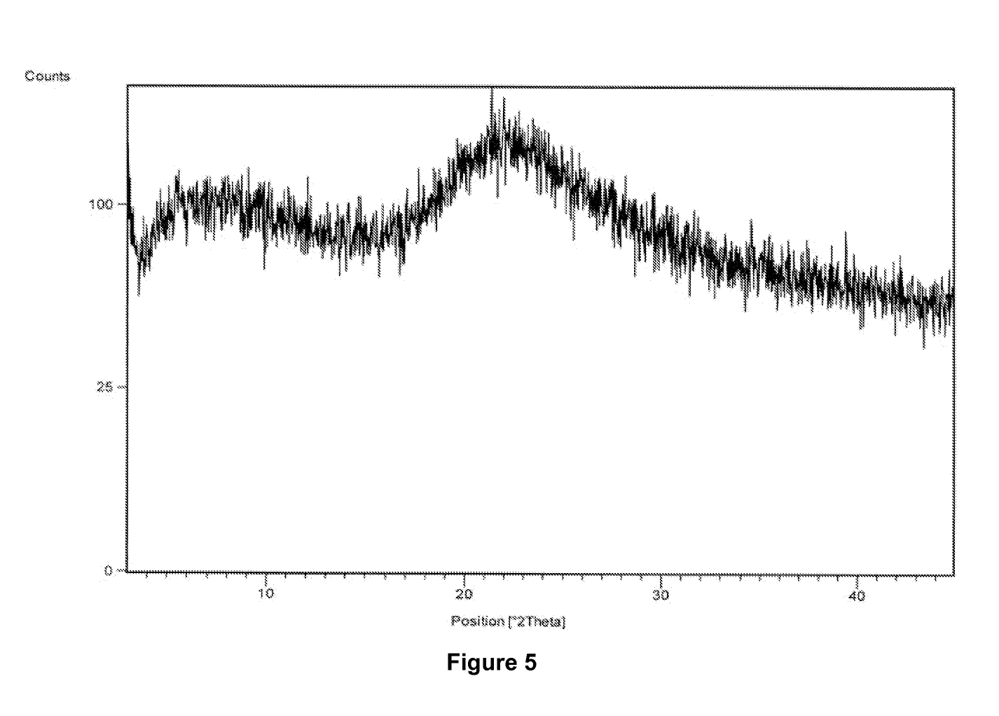

Lorcaserin Hydrochloride (1000 mg) and PVP-K-30 (500 mg) were taken in Methanol (25 ml) and stirred at room temperature to obtain a clear solution. The resulting solution was subjected to distillation under reduced pressure at 60 to 65° C. for 30-45 minutes to obtain amorphous form comprising lorcaserin hydrochloride. PXRD Pattern is shown as FIG. 5.

1. Amorphous lorcaserin hydrochloride.

2. Amorphous lorcaserin hydrochloride according to claim 1, characterized by X-ray powder diffraction pattern substantially as illustrated by FIG. 2.

3. A process for the preparation of amorphous form of lorcaserin hydrochloride according to claim 1, comprising:
a) providing a solution of lorcaserin hydrochloride in a suitable solvent or mixtures thereof; and
b) isolating amorphous form of lorcaserin hydrochloride.

4. An amorphous solid dispersion comprising lorcaserin hydrochloride and one or more pharmaceutically acceptable carriers.

5. The amorphous solid dispersion according to claim 4, characterized by X-ray powder diffraction pattern substantially as illustrated by FIG. 1, FIG. 3 or FIG. 4.

6. The amorphous solid dispersion according to claim 4, wherein pharmaceutically acceptable carriers include, mannitol, lactose, fructose, sorbitol, xylitol, maltodextrin, dextrates, dextrins, lactitol, inositol, trehalose, maltose, raffinose, α-, β- and γ-cyclodextrins, gum arabic, sodium alginate, propylene glycol alginate, agar, gelatin, tragacanth, xanthan gum, starch, lectins, urea, chitosan, chitosan glutamate, hydroxypropyl β-cyclodextrin chitosan, hydroxypropylmethylcellulose (HPMC), hydroxypropylcellulose (HPC), methylcellulose (MC), cellulose acetate phthalate (CAP), hydroxypropylmethylcellulose phthalate (HPMC-P), hydroxylpropyl methylcellulose acetate succinate (HPMC-AS), carboxymethylethylcellulose (CMEC), carboxymethyl cellulose, sodium carboxymethyl cellulose, cellulose acetate butyrate, hydroxyethyl cellulose, ethyl cellulose, co-(lactic/glycolic)copolymers, poly(orthoester), polyvinyl chloride, polyvinyl acetate, ethylene vinyl acetate, carbopols, silicon elastomers, polyacrylic polymers, polyvinylacetal diethylaminoacetate, aminoalkyl methacrylate copolymer E, aminoalkyl methacryl copolymer RS, methacrylic acid copolymer L, methacrylic acid copolymer LD, methacrylic acid copolymer S, carboxylvinyl polymer, polyvinylpyrrolidones (homopolymers or copolymers of N-vinyl pyrrolidone), polyethyleneglycols of various molecular weights, polyethylene-/polypropylene-/polyethylene-oxide block copolymers, polymethacrylates, polyvinylalcohol (PVA) and co-polymers thereof with PVP or with other polymers, polyacrylates, hypromellose phthalates, polyhydric alcohols, polyethylene glycols, polyethylene oxides, polyoxyethylene derivatives, alkyl amines (primary, secondary, and tertiary), aromatic amines, alicyclic amines, cyclic amines, aralkyl amines, hydroxylamine or its derivatives, hydrazine or its derivatives, guanidine or its derivatives; diluents such as starches, dextrin, pullulan, corn starch and potato starch pregelatinized starches; lactose, sucrose, glucose, reduced maltose, mannitol, sorbitol, xylitol, trehalose, powdered cellulose, microcrystalline cellulose, dicalcium phosphate, tricalcium phosphate, crystalline cellulose/carmellose sodium, hydroxypropyl cellulose, magnesium aluminometasilicate, silicon dioxide, or light anhydrous silicic acid; binders such as acacia, guar gum, tragacanth, gelatin, polyvinylpyrrolidones, hydroxypropyl celluloses, hydroxypropyl methylcelluloses, or pregelatinized starches; disintegrants such as hydroxypropyl cellulose, low-substituted hydroxypropyl cellulose, croscarmellose sodium, a starch, methylcellulose, sodium alginate, sodium carboxymethyl starch, carmellose calcium, carmellose sodium, crystalline cellulose and crystalline cellulose/carmellose sodium, sodium starch glycolate, pregelatinized starches, crospovidones, or colloidal silicon dioxide; lubricants such as stearic acid, magnesium stearate, talc, light anhydrous silicic acid, calcium stearate, zinc stearate, magnesium oxide, sodium lauryl sulfate, sodium stearyl fumarate, or magnesium aluminometasilicate; flavoring agents such as sucrose, aspartame, mannitol, dextran, saccharin, menthol, citric acid, tartaric acid, malic acid, ascorbic acid, sweet hydrangea leaves, fennel, ethanol, fructose, xylitol, glycyrrhizinic acid, purified sucrose, L-glutamine, cyclodextrin, peppermint, or methyl salicylate; surfactants such as sodium lauryl sulfate, polysolvate 80, sucrose fatty acid ester, polyoxyl 40 stearate, polyoxyethylene 60 hydrogenated castor oil, sorbitan monostearate, or sorbitan monopalmitate; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, or waxes; film formers; plasticizers; colorants; viscosity enhancers; preservatives; or antioxidants.

7. The amorphous solid dispersion according to claim 4, wherein pharmaceutically acceptable carrier is used in about 0.1, about 0.5, about 1.0, about 2.0, about 3.0, about 4.0, about 5.0, about 6.0, about 7.0, about 8.0, about 9.0 or about 10.0 portions by weight per one portion by weight of lorcaserin hydrochloride.

8. A process for preparing an amorphous solid dispersion according to claim 4, comprising the steps of:
a) dissolving or dispersing lorcaserin hydrochloride and one or more pharmaceutically acceptable carriers in a suitable solvent or mixtures thereof; and
b) isolating an amorphous solid dispersion comprising lorcaserin hydrochloride and one or more pharmaceutically acceptable carriers.

9. A process for preparing an amorphous solid dispersion according to claim 4, comprising the steps of:
(a) dissolving or dispersed lorcaserin hydrochloride under heating in one or more pharmaceutically acceptable carriers; and
(b) cooling down the mixture.

10. A process for preparing an amorphous solid dispersion according to claim 4, comprising the steps of:
(a) dissolving or dispersing lorcaserin hydrochloride and one or more pharmaceutically acceptable carriers in a suitable solvent or mixture of solvents;
(b) spray drying the mixture to obtain the said amorphous solid dispersion.

11. A pharmaceutical composition comprising amorphous lorcaserin hydrochloride and one or more pharmaceutically acceptable carriers.

12. A pharmaceutical composition according to claim 11, wherein pharmaceutically acceptable carriers include, mannitol, lactose, fructose, sorbitol, xylitol, maltodextrin, dextrates, dextrins, lactitol, inositol, trehalose, maltose, raffinose, α-, β- and γ-cyclodextrins, gum arabic, sodium alginate, propylene glycol alginate, agar, gelatin, tragacanth, xanthan gum, starch, lectins, urea, chitosan, chitosan glutamate, hydroxypropyl β-cyclodextrin chitosan, hydroxypropylmethylcellulose (HPMC), hydroxypropylcellulose (HPC), methylcellulose (MC), cellulose acetate phthalate (CAP), hydroxypropylmethylcellulose phthalate (HPMC-P), hydroxylpropyl methylcellulose acetate succinate (HPMC-AS), carboxymethylethylcellulose (CMEC), carboxymethyl cellulose, sodium carboxymethyl cellulose, cellulose acetate butyrate, hydroxyethyl cellulose, ethyl cellulose, co-(lactic/glycolic)copolymers, poly(orthoester), polyvinyl chloride, polyvinyl acetate, ethylene vinyl acetate, carbopols, silicon elastomers, polyacrylic polymers, polyvinylacetal diethylaminoacetate, aminoalkyl methacrylate copolymer E, aminoalkyl methacryl copolymer RS, methacrylic acid copolymer L, methacrylic acid copolymer LD, methacrylic acid copolymer S, carboxylvinyl polymer, polyvinylpyrrolidones (homopolymers or copolymers of N-vinyl pyrrolidone), polyethyleneglycols of various molecular weights, polyethylene-/polypropylene-/polyethylene-oxide block copolymers, polymethacrylates, polyvinylalcohol (PVA) and co-polymers thereof with PVP or with other polymers, polyacrylates, hypromellose phthalates, polyhydric alcohols, polyethylene glycols, polyethylene oxides, polyoxyethylene derivatives, alkyl amines (primary, secondary, and tertiary), aromatic amines, alicyclic amines, cyclic amines, aralkyl amines, hydroxylamine or its derivatives, hydrazine or its derivatives, guanidine or its derivatives; diluents such as starches, dextrin, pullulan, corn starch and potato starch pregelatinized starches; lactose, sucrose, glucose, reduced maltose, mannitol, sorbitol, xylitol, trehalose, powdered cellulose, microcrystalline cellulose, dicalcium phosphate, tricalcium phosphate, crystalline cellulose/carmellose sodium, hydroxypropyl cellulose, magnesium aluminometasilicate, silicon dioxide, or light anhydrous silicic acid; binders such as acacia, guar gum, tragacanth, gelatin, polyvinylpyrrolidones, hydroxypropyl celluloses, hydroxypropyl methylcelluloses, or pregelatinized starches; disintegrants such as hydroxypropyl cellulose, low-substituted hydroxypropyl cellulose, croscarmellose sodium, a starch, methylcellulose, sodium alginate, sodium carboxymethyl starch, carmellose calcium, carmellose sodium, crystalline cellulose and crystalline cellulose/carmellose sodium, sodium starch glycolate, pregelatinized starches, crospovidones, or colloidal silicon dioxide; lubricants such as stearic acid, magnesium stearate, talc, light anhydrous silicic acid, calcium stearate, zinc stearate, magnesium oxide, sodium lauryl sulfate, sodium stearyl fumarate, or magnesium aluminometasilicate; flavoring agents such as sucrose, aspartame, mannitol, dextran, saccharin, menthol, citric acid, tartaric acid, malic acid, ascorbic acid, sweet hydrangea leaves, fennel, ethanol, fructose, xylitol, glycyrrhizinic acid, purified sucrose, L-glutamine, cyclodextrin, peppermint, or methyl salicylate; surfactants such as sodium lauryl sulfate, polysolvate 80, sucrose fatty acid ester, polyoxyl 40 stearate, polyoxyethylene 60 hydrogenated castor oil, sorbitan monostearate, or sorbitan monopalmitate; complex forming agents such as various grades of cyclodextrins and resins; release rate controlling agents such as hydroxypropyl celluloses, hydroxymethyl celluloses, hydroxypropyl methylcelluloses, ethylcelluloses, methylcelluloses, various grades of methyl methacrylates, or waxes; film formers; plasticizers; colorants; viscosity enhancers; preservatives; or antioxidants.
